(19) United States
(12) Patent Application Publication
(10) Pub. No.: US 2016/0375019 A1
(43) Pub. Date: Dec. 29, 2016

(54) TREATMENT OF CHRONIC GRAFT VERSUS HOST DISEASE WITH SYK INHIBITORS

(71) Applicant: Gilead Sciences, Inc., Foster City, CA (US)

(72) Inventors: Julie A. Di Paolo, San Francisco, CA (US); Joseph Haw-Ling Lin, San Maeo, CA (US); Shao-Lee Lin, North Chicago, IL (US)

(21) Appl. No.: 15/133,041

(22) Filed: Apr. 19, 2016

Related U.S. Application Data
(60) Provisional application No. 62/150,691, filed on Apr. 21, 2015.

(51) Int. Cl. A61K 31/4985; A61K 31/5377; A61K 45/06

(52) U.S. Cl. CPC A61K 31/4985; A61K 45/06; A61K 31/5377

(57) ABSTRACT

The present disclosure provides methods of utilizing Syk inhibiting compounds in the treatment for graft versus host disease (GVHD) in a human, including acute graft versus host disease (aGVHD) and chronic graft versus host disease (cGVHD), including the use of compounds selected from the group consisting of the formulas below:

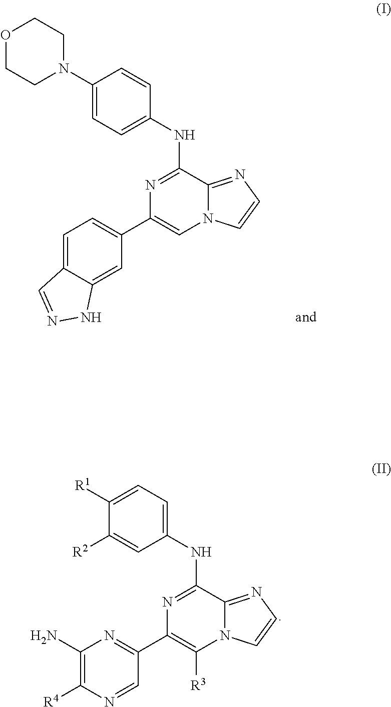

### CROSS-REFERENCES TO RELATED APPLICATIONS

This patent application claims the benefit under 35 U.S.C. §119(e) of U.S. Provisional Patent Application No. 62/150,691, filed Apr. 21, 2015. The foregoing patent application is incorporated herein by reference in its entirety.

### FIELD

The present disclosure relates to methods of utilizing Syk inhibiting compounds in the treatment for graft versus host disease (GVHD) in a human, including acute graft versus host disease (aGVHD) and chronic graft versus host disease (cGVHD).

### BACKGROUND

Protein kinases, the largest family of human enzymes, encompass well over 500 human proteins. Spleen Tyrosine Kinase (Syk) is a member of the Syk family of tyrosine kinases, and is a regulator of early B-cell development as well as mature B-cell activation, signaling, and survival.

Acute Graft Versus Host Disease (aGVHD), also known as fulminant Graft Versus Host Disease, generally presents symptoms within the first 100 days following allogenic hematopoietic stem cell transplantation and is generally characterized by selective damage to the skin, liver, mucosa, and gastrointestinal tract. Chronic Graft Versus Host Disease (cGVHD) occurs in recipients of allogeneic hematopoietic stem cell transplant (HSCT). GVHD is considered chronic when it occurs >100 days post-transplant, though aspects of cGVHD may manifest themselves prior to the 100 day point and overlap with elements of aGVHD. The disease has a cumulative incidence of 35-70% of transplanted patients, and has an annual incidence of approximately 3,000-5,000 and a prevalence of approximately 10,000 in the US. cGVHD is difficult to treat and is associated with worse outcomes compared to those without cGVHD. Current standard of care includes a variety of approaches including systemic corticosteroids often combined with calcineurin inhibitors, mTOR inhibitors, mycophenylate mofetil, or rituximab. Despite treatment, response rates are poor (40-50%) and cGVHD is associated with significant morbidity such as serious infection and impaired quality of life; the 5-year mortality is 30-50% (Blazar et al., Nature Reviews Immunology 12, 443-458, June 2012).

Human and animal models have demonstrated that aberrant B-lymphocyte signaling and survival is important in the pathogenesis of cGVHD. B-cell targeted drugs, including SYK inhibitors (fostamatinib-13 Sarantopoulos et al., Biology of Blood and Marrow Transplantation, 21(2015) S11-S18) and BTK inhibitors (ibrutinib—Nakasone et al., Int. J. Hematol.—27 Mar. 2015), have been shown to selectively reduce the function and frequency of aberrant GVHD B-cell populations ex vivo.

There remains a need for new methods, pharmaceutical compositions, and regimens for the treatment of GVHD, including aGVHD and cGVHD.

### SUMMARY

Accordingly, the present disclosure provides compounds that function as Syk inhibitors in a method for treating graft versus host disease (GVHD) in a human, including acute graft versus host disease (aGVHD) and chronic graft versus host disease (cGVHD), the method comprising administering to the human in need thereof a pharmaceutically effective amount of a Syk inhibitor. It is understood that the terms Syk inhibiting compounds, Syk inhibitor compounds, and Syk inhibitors are synonymous as used herein.

Examples of Syk inhibiting compounds that may be used independently in these methods of treating cGVHD in a human include those of selected from the group consisting of the structures below, or a pharmaceutically acceptable salt or co-crystal thereof:

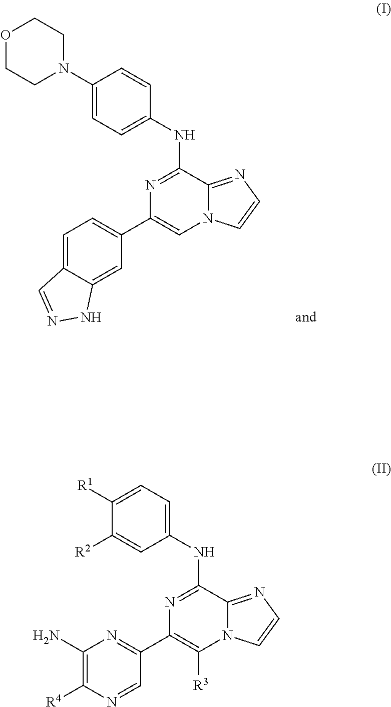

wherein, in Formula (II):
R¹ is selected from the group consisting of

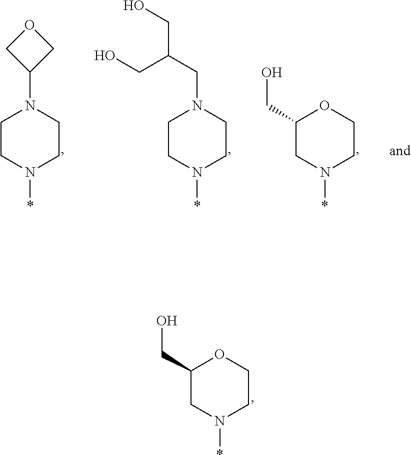

wherein * indicates the carbon atom of the indicated phenyl ring of Formula 1 to which R¹ is attached;
R² is H or 2-hydroxyethoxyl;
R³ is H or methyl; and
R⁴ is H or methyl.

### BRIEF DESCRIPTION OF THE DRAWINGS

FIG. 1 represents raw values for apoptosis induction seen in human cGVHD and non-cGVHD B cells treated with entospletinib.

FIG. 2 represents values for increased apoptosis in human cGVHD B cells treated with entospletinib.

### DETAILED DESCRIPTION

One embodiment provides a method for treating graft versus host disease (GVHD) in a human, the method comprising administering to the human in need thereof a pharmaceutically effective amount of a compound of Formula (II):

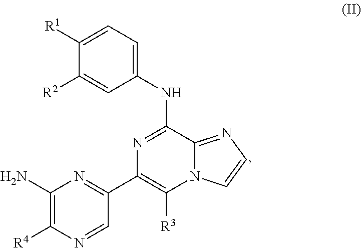

wherein R¹, R², R³, and R⁴ are as defined above, or a pharmaceutically acceptable salt or co-crystal thereof. Preparation of compounds of Formula (II) can be seen in US 2015/0175616 A1 (Blomgren et al.)

Another embodiment provides a method for treating acute graft versus host disease (aGVHD) in a human, the method comprising administering to the human in need thereof a pharmaceutically effective amount of a compound of Formula (II), as defined above, or a pharmaceutically acceptable salt or co-crystal thereof.

A further embodiment provides a method for treating chronic graft versus host disease (cGVHD) in a human, the method comprising administering to the human in need thereof a pharmaceutically effective amount of a compound of Formula (II), as defined above, or a pharmaceutically acceptable salt or co-crystal thereof.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), each of R², R³, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is H, R³ is methyl, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is H, R³ is H, and R⁴ is methyl, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is 2-hydroxyethoxyl, R³ is methyl, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is 2-hydroxyethoxyl, R³ is methyl, and R⁴ is H, and R¹ is as defined above.

Within each reference to an embodiment herein, including to a method of treatment, pharmaceutical composition, or therapeutic regimen, concerning a compound of Formula (II), or a pharmaceutically acceptable salt or co-crystal thereof, it is understood that within each embodiment there is a further embodiment in which, in the compound of Formula (II), R² is 2-hydroxyethoxyl, R³ is H, and R⁴ is methyl, and R¹ is as defined above.

Within each reference to an embodiment herein, including reference to a method of treatment, pharmaceutical composition, or therapeutic regimen, concerning a compound of Formula (II), it is understood that within each there are separate treatments, pharmaceutical compositions, or therapeutic regimens in which the compound of Formula (II) comprises, individually:

6-(6-amino-5-methylpyrazin-2-yl)-N-(4-(4-(oxetan-3-yl)piperazn-1-yl)phenyl)imidazo[1,2-a]pyrazin-8-amine;

6-(6-aminopyrazin-2-yl)-N-(4-(4-(oxetan-3-yl)piperazin-1-yl)phenyl)imidazo[1,2-a]pyrazin-8-amine;

(R)-(4-(4-((6-(6-aminopyrazin-2-yl)imidazo[1,2-a]pyrazin-8-yl)amino)phenyl)morpholin-2-yl)methanol;

6-(6-aminopyrazin-2-yl)-5-methyl-N-(4-(4-(oxetan-3-yl)piperazin-1-yl)phenyl)imidazo[1,2-a]pyrazin-8-amine;

2-(5-((6-(6-aminopyrazin-2-yl)imidazo[1,2-a]pyrazin-8-yl)amino)-2-(4-(oxetan-3-yl)piperazin-1-yl)phenoxy)ethanol;

2-((4-(4-((6-(6-aminopyrazin-2-yl)imidazo[1,2-a]pyrazin-8-yl)amino)phenyl)piperazin-1-yl)methyl)propane-1,3-diol; or

2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol;

a pharmaceutically acceptable salt of co-crystal thereof.

For each of the embodiments disclosed herein, including the methods of treatment, pharmaceutical compositions, kits, regimens, and other uses concerning a compound of Formula (I) or of Formula (II), including the specific examples of compounds within Formula (II) disclosed herein, it is understood that reference to a compound of Formula (I) or of Formula (II) or a pharmaceutically acceptable salt of co-crystal thereof, also includes pharmaceutically acceptable esters, pharmaceutically acceptable solvates, hydrates, isomers (including optical isomers, racemates, or other mixtures thereof), tautomers, isotopes, polymorphs, and pharmaceutically acceptable prodrugs of such compounds.

A separate embodiment provides a method for treating graft versus host disease (GVHD) in a human, the method comprising administering to the human in need thereof a pharmaceutically effective amount of 6-(1H-indazol-6-yl)-N-(4-morpholinophenyl)imidazo[1,2-a]pyrazin-8-amine (Formula I), having the structure:

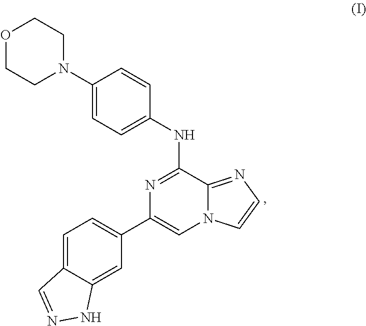 [SMILES: c1cn2cc(-c3ccc4cn[nH]c4c3)nc(Nc3ccc(N4CCOCC4)cc3)c2n1]

or a pharmaceutically acceptable salt or co-crystal thereof. The compound of Formula I, above, may also be referred to as entospletinib or GS-9973.

Another embodiment provides a method for treating acute graft versus host disease (aGVHD) in a human, the method comprising administering to the human in need thereof a pharmaceutically effective amount of 6-(1H-indazol-6-yl)-N-(4-morpholinophenyl)imidazo[1,2-a]pyrazin-8-amine (Formula I), or a pharmaceutically acceptable salt or co-crystal thereof.

A further embodiment provides a method for treating chronic graft versus host disease (cGVHD) in a human, the method comprising administering to the human in need thereof a pharmaceutically effective amount of 6-(1H-indazol-6-yl)-N-(4-morpholinophenyl)imidazo[1,2-a]pyrazin-8-amine (Formula I), or a pharmaceutically acceptable salt or co-crystal thereof.

One embodiment provides the use of a compound of Formula (I) or of Formula (II): or a pharmaceutically acceptable salt or co-crystal thereof:

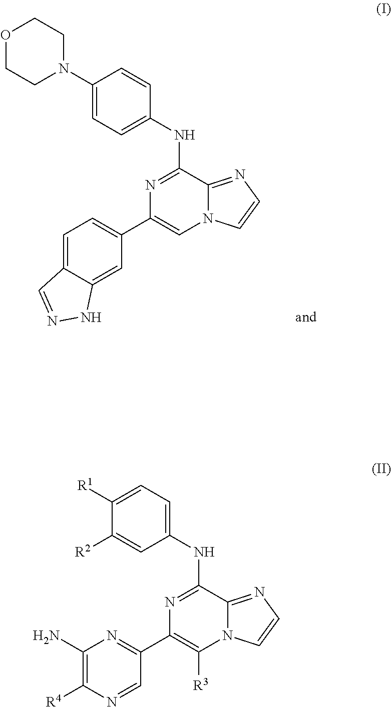

wherein, in Formula (II):
R¹ is selected from the group consisting of

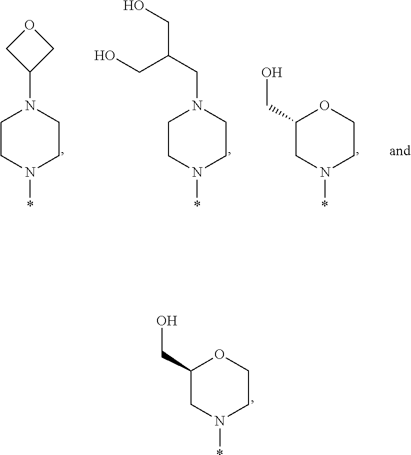

wherein * indicates the carbon atom of the indicated phenyl ring of Formula 1 to which R¹ is attached;
R² is H or 2-hydroxyethoxyl;
R³ is H or methyl; and
R⁴ is H or methyl;
   in the manufacture of a medicament for the treatment of graft versus host disease (GVHD) in a human.

An additional embodiment provides a method for inhibiting the onset of symptoms of GVHD, including aGVHD and cGVHD, the method comprising administering to a human recipient of a transplantation of allogenic hematopoietic stem cells, the method comprising administering to the human in need thereof a pharmaceutically effective amount of 6-(1H-indazol-6-yl)-N-(4-morpholinophenyl)imidazo[1,2-a]pyrazin-8-amine (Formula I), or a pharmaceutically acceptable salt or co-crystal thereof. As such, an additional embodiment provides a method for inhibiting the onset of symptoms of aGVHD, the method comprising administering to a human recipient of a transplantation of allogenic hematopoietic stem cells, the method comprising administering to the human in need thereof a pharmaceutically effective amount of 6-(1H-indazol-6-yl)-N-(4-morpholinophenyl)imidazo[1,2-a]pyrazin-8-amine (Formula I), or a pharmaceutically acceptable salt or co-crystal thereof. As such, an additional embodiment provides a method for inhibiting the onset of symptoms of cGVHD, the method comprising administering to a human recipient of a transplantation of allogenic hematopoietic stem cells, the method comprising administering to the human in need thereof a pharmaceutically effective amount of 6-(1H-indazol-6-yl)-N-(4-morpholinophenyl)imidazo[1,2-a]pyrazin-8-amine (Formula I), or a pharmaceutically acceptable salt or co-crystal thereof. Within each of the embodiments described herein including the use of a compound of Formula (I), there is a further embodiment in which the compound of Formula (I) is used as a mesylate salt. Within each of the embodiments described herein including the use of a compound of Formula (I), there is a further embodiment in which the compound of Formula (I) is used as a bis-mesylate salt. Within each of the embodiments described herein including the use of a compound of Formula (I), there is a further embodiment in which the compound of Formula (I) is used as a bis-mesylate salt of Form 3, described herein. Within each of the embodiments described herein including the use of a compound of Formula (I), there is also a further embodiment in which the compound of Formula (I) is used as a bis-mesylate salt of Form 7, described herein. Mesylate salts of the compound of Formula (I), including Form 3 and Form 7, are taught by Elford et al, U.S. Pat. Appin. Publ. 2015/0038505 A1, the contents of which are incorporated herein by reference.

Another embodiment provides a method for inhibiting the onset of symptoms of GVHD, including aGVHD and cGVHD, the method comprising administering to a human recipient of a transplantation of allogenic hematopoietic stem cells a pharmaceutically effective amount of a compound of Formula (II), or a pharmaceutically acceptable salt or co-crystal thereof An embodiment provides a method for inhibiting the onset of symptoms of aGVHD, the method comprising administering to a human recipient of a transplantation of allogenic hematopoietic stem cells a pharmaceutically effective amount of a compound of Formula (II), or a pharmaceutically acceptable salt or co-crystal thereof Another embodiment provides a method for inhibiting the onset of symptoms of cGVHD, the method comprising administering to a human recipient of a transplantation of allogenic hematopoietic stem cells a pharmaceutically effective amount of a compound of Formula (II), or a pharmaceutically acceptable salt or co-crystal thereof.

Another embodiment provides a method of treating GVHD in a human, including aGVHD and cGVHD, the method comprising administering to the human in need thereof a pharmaceutically effective amount of a compound of Formula (I) or of Formula (II), or a pharmaceutically acceptable salt or co-crystal form thereof, in combination with a pharmaceutically effective amount of another agent useful in treating GVHD in a human, including aGVHD and cGVHD. A further embodiment provides a method of treating aGVHD in a human, the method comprising administering to the human in need thereof a pharmaceutically effective amount of a compound of Formula (I) or of Formula (II), or a pharmaceutically acceptable salt or co-crystal form thereof, in combination with a pharmaceutically effective amount of another agent useful in treating aGVHD in a human. Another embodiment provides a method of treating cGVHD in a human, the method comprising administering to the human in need thereof a pharmaceutically effective amount of a compound of Formula (I) or of Formula (II), or a pharmaceutically acceptable salt or co-crystal form thereof, in combination with a pharmaceutically effective amount of another agent useful in treating cGVHD in a human. Agents useful for treating GVHD include immunosuppressive agents, antiproliferatives (e.g., antibiotics), anti-inflammatories, pain relievers, etc.

Another embodiment provides a method for inhibiting the onset of symptoms of GVHD, including aGVHD and cGVHD, the method comprising administering to a human recipient of a transplantation of allogenic hematopoietic stem cells a pharmaceutically effective amount of a pharmaceutically effective amount of a compound of Formula (I) or of Formula (II), or a pharmaceutically acceptable salt or co-crystal form thereof, in combination with a pharmaceutically effective amount of another agent useful in treating GVHD in a human, including aGVHD and cGVHD.

Another embodiment provides a method for inhibiting the onset of symptoms of aGVHD in a human recipient of a transplantation of allogenic hematopoietic stem cells, the method comprising administering to the human a pharmaceutically effective amount of a pharmaceutically effective amount of a compound of Formula (I), or a pharmaceutically acceptable salt or co-crystal form thereof, in combination with a pharmaceutically effective amount of another agent useful in treating aGVHD.

Another embodiment provides a method for inhibiting the onset of symptoms of cGVHD in a human recipient of a transplantation of allogenic hematopoietic stem cells, the method comprising administering to the human a pharmaceutically effective amount of a pharmaceutically effective amount of a compound of Formula (I), or a pharmaceutically acceptable salt or co-crystal form thereof, in combination with a pharmaceutically effective amount of another agent useful in treating cGVHD.

Another embodiment provides a method for inhibiting the onset of symptoms of aGVHD in a human recipient of a transplantation of allogenic hematopoietic stem cells, the method comprising administering to the human a pharmaceutically effective amount of a pharmaceutically effective amount of a compound of Formula (II), or a pharmaceutically acceptable salt or co-crystal form thereof, in combination with a pharmaceutically effective amount of another agent useful in treating aGVHD.

Another embodiment provides a method for inhibiting the onset of symptoms of cGVHD in a human recipient of a transplantation of allogenic hematopoietic stem cells, the method comprising administering to the human a pharmaceutically effective amount of a pharmaceutically effective amount of a compound of Formula (II), or a pharmaceutically acceptable salt or co-crystal form thereof, in combination with a pharmaceutically effective amount of another agent useful in treating cGVHD.

Examples of agents that may be combined in the methods herein with the compounds of Formulas (I) and (II), or a pharmaceutically acceptable salt or co-crystal form thereof, include steroids, such as prednisone and methylprednisone, oral nonabsorbable corticosteroids, such as budesonide or beclomethasone diproprionate, immune modulators, such as cyclosporine (Neoral®, Sandimmune®), tacrolimus (Prograf®), sirolimus (Rapamune®), mycophenolate mofetil (CellCept®), tilomisole, imuthiol, antithymocyte globulin (ATG), anti-TNF agents, azathioprine (or other inosine 5′-monophosphate dehydrogenase inhibitors), azodiacarbonide, bisindolyl maleimide VIII, brequinar, chlorambucil, CTLA4-Ig, corticosteroids, cyclophosphamide, deoxyspergualin, dexamethasone, glucocorticoids, leflunomide, mercaptopurine, 6-mercaptopurine (6-MP), methotrexate, methylprednisolone, mizoribine, mizoribine monophosphate, muromonab CD3, mycophenolate mofetil, OKT3, prednisone, sirolimus, rapamycin, rho (D) immune globin, tacrolimus (FK506), vitamin D analogs (e.g., MC1288), etc., monoclonal antibodies, such as daclizumab (Zenapax®), infliximab (Remicade®), rituximab (Rituxan®, MabThera®, or Zytux®), tocilizumab (Actemra®), and alemtuzumab (Campath®), methotrexate, antithymocyte globulin (rabbit ATG, Thymoglobulin®), Denileukin diftitox (Ontak®), Campath-1H, keratinocyte growth factor (KGF), abatacept (Orencia®), remestemcel-L (Prochyma1®), suberoylanilide hydroxamic acid (SAHA), pentostatin (deoxycoformycin, Nipent®), thalidomide (Thalomid®), imatinib mesylate (Gleevec®), cyclophosphamide, fludarabine, OKT3 (Muromorab CO3®, Orthoclone®), melphalan, thiopeta, and ATGAM® (lymphocyte immune globulin, anti-thymocyte, globulin [equine]sterile solution).

It will be understood that the methods referenced above may comprise the administration to the human in need of GVHD treatment a pharmaceutically effective amount of a compound of Formula (I) or Formula (II), or a pharmaceutically acceptable salt or co-crystal form thereof, in combination with one or more additional agents useful in treating GVHD. For instance a pharmaceutically effective amount of a compound of Formula (I) or Formula (II), or a pharmaceutically acceptable salt or co-crystal form thereof may be combined with administration of pharmaceutically effective amount of one or more steroids such as prednisone, methylprednisone, budesonide or beclomethasone diproprionate, and a pharmaceutically effective amount of an immune modulator such as cyclosporine (Neoral®, Sandimmune®), tacrolimus (Prograf®), sirolimus (Rapamune®), or mycophenolate mofetil (CellCept®).

It is also understood that each of the agents administered individually or combined in a combination therapy or regimen may be administered at an initial dose that may then over time be reduced by a medical professional to reach a lower effective dose. For instance, in the combinations and regimens herein, systemic glucocorticosteroids (corticosteroids), such as prednisone and methyl prednisone may be administered to a human patient at a dose of from about 1-2 mg/kg/day. Initial daily doses for mTOR agents include sirolimus at 2-40 mg given once daily and everolimus at 0.25-1 mg given twice daily. Initial daily doses for calcineurin agents include tacrolimus at from about 0.025-0.2 mg/kg/day and cyclosporine at from about 2.5-9 mg/kg/day. Mycophenolate mofetil (CellCept®) may be administered at an initial daily dose of about 250-3,000 mg/day. Each of these agents may be administered in combination with a pharmaceutically effective amount of a Syk inhibitor as described herein following hematopoietic cell transplant. In different embodiments herein, agents useful in treating GVHD may be administered topically to a human in need of such treatment, such as in the form of a topical ointment or cream or in an eye drop formulation.

“Also provided are uses of the compound of Formula I, or a pharmaceutically acceptable salt or co-crystal thereof, in the manufacture of a medicament for the treatment of graft versus host disease (GVHD) in a human, including acute graft versus host disease (aGVHD) and chronic graft versus host disease (cGVHD). Also provided are uses of the compound of Formula (II), or a pharmaceutically acceptable salt or co-crystal thereof, in the manufacture of a medicament for the treatment of graft versus host disease (GVHD) in a human, including acute graft versus host disease (aGVHD) and chronic graft versus host disease (cGVHD).

Examples of forms of the compound of Formula (I) that may be used in the methods and combination therapies described herein include those known in the art, including those described in U.S. 2015/0038505 and WO 2015/017460, the contents of which are incorporated herein by reference. Such forms include a bis-mesylate form of a compound of Formula (I), or a hydrate thereof, and include polymorph Form 3 and polymorph Form 7. Within each of the embodiments described herein concerning methods of treatment, pharmaceutical compositions, kits, regimens, and other uses wherein the Syk compound utilized is a compound of Formula (I), entospletinib, there is a further embodiment in which the compound of Formula (I) is a bis-mesylate form of polymorph Form 3. In each of the embodiments described herein concerning methods of treatment, pharmaceutical compositions, kits, regimens, and other uses wherein the Syk compound utilized is a compound of Formula (I), entospletinib, there is also a further embodiment in which the compound of Formula (I) is a bis-mesylate form of polymorph Form 7.

### Definitions

As used herein, by “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable vehicles (e.g., carriers, adjuvants, and/or other excipients) have preferably met the required standards of toxicological and manufacturing testing and/or are included on the Inactive Ingredient Guide prepared by the U.S. Food and Drug administration.

“Pharmaceutically acceptable salts” include, for example, salts with inorganic acids and salts with an organic acid. Examples of salts may include hydrochlorate, phosphate, diphosphate, hydrobromate, sulfate, sulfinate, nitrate, malate, maleate, fumarate, tartrate, succinate, citrate, acetate, lactate, mesylate, p-toluenesulfonate, 2-hydroxyethylsulfonate, benzoate, salicylate, stearate, and alkanoate (such as acetate, HOOC—(CH₂)ₙ—COOH where n is 0-4). In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

The terms “effective amount”, “pharmaceutically effective amount”, and “therapeutically effective amount” refer to an amount that may be effective to elicit the desired biological or medical response, including the amount of a compound that, when administered to a subject for treating a disease, is sufficient to effect such treatment for the disease. The effective amount will vary depending on the compound, the disease and its severity and the age, weight, etc., of the subject to be treated. The effective amount can include a range of amounts. A pharmaceutically effective amount includes amounts of an agent which are effective when combined with other agents.

“Treatment” or “treating” is an approach for obtaining beneficial or desired results including clinical results. Beneficial or desired clinical results may include one or more of the following:
(i) decreasing one more symptoms resulting from the disease;
(ii) diminishing the extent of the disease and/or stabilizing the disease (e.g., delaying the worsening of the disease);
(iii) delaying the spread of the disease;
(iv) delaying or slowing the onset or recurrence of the disease and/or the progression of the disease;
(v) ameliorating the disease state and/or providing a remission (whether partial or total) of the disease and/or decreasing the dose of one or more other medications required to treat the disease;
(vi) increasing the quality of life, and/or
(vii) prolonging survival.

“Delaying” the development of a disease or condition means to defer, hinder, slow, retard, stabilize, and/or postpone development of the disease or condition. This delay can be of varying lengths of time, depending on the history of the disease or condition, and/or subject being treated. A method that “delays” development of a disease or condition is a method that reduces probability of disease or condition development in a given time frame and/or reduces the extent of the disease or condition in a given time frame, when compared to not using the method. Such comparisons are typically based on clinical studies, using a statistically significant number of subjects. Disease or condition development can be detectable using standard methods, such as routine physical exams, mammography, imaging, or biopsy. Development may also refer to disease or condition progression that may be initially undetectable and includes occurrence, recurrence, and onset.

For use in the methods described herein, the compound of Formula (I) or Formula (II), or a pharmaceutically acceptable salt or co-crystal thereof, may be present in a pharmaceutical composition comprising the compound of Formula (I) or Formula (II), or a pharmaceutically acceptable salt or co-crystal thereof, and at least one pharmaceutically acceptable vehicle. Pharmaceutically acceptable vehicles may include pharmaceutically acceptable carriers, adjuvants and/or other excipients, and other ingredients can be deemed pharmaceutically acceptable insofar as they are compatible with other ingredients of the formulation and not deleterious to the recipient thereof.

The pharmaceutical compositions of the compound of Formula (I) or Formula (II), or a pharmaceutically acceptable salt or co-crystal thereof, described herein can be manufactured using any conventional method, e.g., mixing, dissolving, granulating, dragee-making, levigating, emulsifying, encapsulating, entrapping, melt-spinning, spray-drying, or lyophilizing processes. An optimal pharmaceutical formulation can be determined by one of skill in the art depending on the route of administration and the desired dosage. Such formulations can influence the physical state, stability, rate of in vivo release, and rate of in vivo clearance of the administered agent. Depending on the condition being treated, these pharmaceutical compositions can be formulated and administered systemically or locally.

The term “carrier” refers to diluents, disintegrants, precipitation inhibitors, surfactants, glidants, binders, lubricants, and other excipients and vehicles with which the compound is administered. Carriers are generally described herein and also in “Remington's Pharmaceutical Sciences” by E. W. Martin. Examples of carriers include, but are not limited to, aluminum monostearate, aluminum stearate, carboxymethylcellulose, carboxymethylcellulose sodium, crospovidone, glyceryl isostearate, glyceryl monostearate, hydroxyethyl cellulose, hydroxyethyl cellulose, hydroxymethyl cellulose, hydroxyoctacosanyl hydroxystearate, hydroxypropyl cellulose, hydroxypropyl cellulose, hydroxypropyl methylcellulose, lactose, lactose monohydrate, magnesium stearate, mannitol, microcrystalline cellulose, poloxamer 124, poloxamer 181, poloxamer 182, poloxamer 188, poloxamer 237, poloxamer 407, povidone, silicon dioxide, colloidal silicon dioxide, silicone, silicone adhesive 4102, and silicone emulsion. It should be understood, however, that the carriers selected for the pharmaceutical compositions, and the amounts of such carriers in the composition, may vary depending on the method of formulation (e.g., dry granulation formulation, solid dispersion formulation).

The term “diluent” generally refers to a substance used to dilute the compound of interest prior to delivery. Diluents can also serve to stabilize compounds. Examples of diluents may include starch, saccharides, disaccharides, sucrose, lactose, polysaccharides, cellulose, cellulose ethers, hydroxypropyl cellulose, sugar alcohols, xylitol, sorbitol, maltitol, microcrystalline cellulose, calcium or sodium carbonate, lactose, lactose monohydrate, dicalcium phosphate, cellulose, compressible sugars, dibasic calcium phosphate dehydrate, mannitol, microcrystalline cellulose, and tribasic calcium phosphate.

The term “disintegrant” generally refers to a substance which, upon addition to a solid preparation, facilitates its break-up or disintegration after administration and permits the release of an active ingredient as efficiently as possible to allow for its rapid dissolution. Examples of disintegrants may include maize starch, sodium starch glycolate, croscarmellose sodium, crospovidone, microcrystalline cellulose, modified corn starch, sodium carboxymethyl starch, povidone, pregelatinized starch, and alginic acid.

The term “precipitation inhibitors” generally refers to a substance that prevents or inhibits precipitation of the active agent from a supersaturated solution. One example of a precipitation inhibitor includes hydroxypropylmethylcellulose (HPMC).

The term “surfactants” generally refers to a substance that lowers the surface tension between a liquid and a solid that could improve the wetting of the active agent or improve the solubility of the active agent. Examples of surfactants include poloxamer and sodium lauryl sulfate.

The term “glidant” generally refers to substances used in tablet and capsule formulations to improve flow-properties during tablet compression and to produce an anti-caking effect. Examples of glidants may include colloidal silicon dioxide, talc, fumed silica, starch, starch derivatives, and bentonite.

The term “binder” generally refers to any pharmaceutically acceptable film which can be used to bind together the active and inert components of the carrier together to maintain cohesive and discrete portions. Examples of binders may include hydroxypropylcellulose, hydroxypropylmethylcellulose, povidone, copovidone, and ethyl cellulose.

The term “lubricant” generally refers to a substance that is added to a powder blend to prevent the compacted powder mass from sticking to the equipment during the tableting or encapsulation process. A lubricant can aid the ejection of the tablet form the dies, and can improve powder flow. Examples of lubricants may include magnesium stearate, stearic acid, silica, fats, calcium stearate, polyethylene glycol, sodium stearyl fumarate, or talc; and solubilizers such as fatty acids including lauric acid, oleic acid, and C₈/C₁₀ fatty acid.

In the methods provided herein, the compound of Formula (I) or of Formula (II), or a pharmaceutically acceptable salt or co-crystal thereof, or a pharmaceutical composition thereof, is administered in a therapeutically effective amount to achieve its intended purpose. Determination of a therapeutically effective amount is well within the capability of those skilled in the art, especially in light of the detailed disclosure provided herein. In some embodiments (methods of treating GVHD), a therapeutically effective amount of the compound of Formula (I) or of Formula (II), or a pharmaceutically acceptable salt or co-crystal thereof, may (i) reduce the severity of GVHD; (ii) slow the onset of symptoms of GVHD; (iii) inhibit, retard, slow to some extent, and preferably stop the spread of GVHD symptoms in the recipient's body; (iv) delay occurrence and/or recurrence of symptoms of GVHD; and/or (v) relieve to some extent one or more of the symptoms associated with the GVHD. In various embodiments, the amount is sufficient to ameliorate, palliate, lessen, and/or delay one or more of symptoms of GVHD, including aGVHD and cGVHD.

The therapeutically effective amount may vary depending on the subject, and disease or condition being treated, the weight and age of the subject, the severity of the disease or condition, and the manner of administering, which can readily be determined by one or ordinary skill in the art.

The dosing regimen of the compound of Formula (I) or of Formula (II), or a pharmaceutically acceptable salt or co-crystal thereof, in the methods provided herein may vary depending upon the indication, route of administration, and severity of the condition, for example. Depending on the route of administration, a suitable dose can be calculated according to body weight, body surface area, or organ size. The final dosing regimen is determined by the attending physician in view of good medical practice, considering various factors that modify the action of drugs, e.g., the specific activity of the compound, the identity and severity of the disease state, the responsiveness of the subject, the age, condition, body weight, sex, and diet of the subject, and the severity of any infection. Additional factors that can be taken into account include time and frequency of administration, drug combinations, reaction sensitivities, and tolerance/response to therapy. Further refinement of the doses appropriate for treatment involving any of the formulations mentioned herein is done routinely by the skilled practitioner without undue experimentation, especially in light of the dosing information and assays disclosed, as well as the pharmacokinetic data observed in human clinical trials. Appropriate doses can be ascertained through use of established assays for determining concentration of the agent in a body fluid or other sample together with dose response data.

The formulation and route of administration chosen may be tailored to the individual subject, the nature of the condition to be treated in the subject, and generally, the judgment of the attending practitioner.

The pharmaceutically effective amount or therapeutically effective amount of the compound of Formula (I) or of Formula (II), or a pharmaceutically acceptable salt or co-crystal thereof, may be provided in a single dose or multiple doses to achieve the desired treatment endpoint. As used herein, “dose” refers to the total amount of an active ingredient (e.g., the compound of Formula (I) or of Formula (II), or a pharmaceutically acceptable salt or co-crystal thereof,) to be taken each time by a subject (e.g., a human). The dose administered, for example for oral administration described above, may be administered once daily (QD), twice daily (BID), three times daily, four times daily, or more than four times daily. In some embodiments, the dose of a compound of Formula (I) or of Formula (II), or a pharmaceutically acceptable salt or co-crystal thereof, is administered once daily. In some embodiments, the dose of a compound of Formula (I) or of Formula (II), or a pharmaceutically acceptable salt or co-crystal thereof, is administered twice daily.

In some embodiments, exemplary doses of the compound of Formula (I) or of Formula (II), or a pharmaceutically acceptable salt or co-crystal thereof, for a human subject may be from about 1 mg to about 5000 mg, about 1 mg to about 4000 mg, about 1 mg to about 3000 mg, about 1 mg to about 2000 mg, about 2 mg to about 2000 mg, about 5 mg to about 2000 mg, about 10 mg to about 2000 mg, about 1 mg to about 1000 mg, about 2 mg to about 1000 mg, about 5 mg to about 1000 mg, about 10 mg to about 1000 mg, about 25 mg to about 1000 mg, about 50 mg to about 1000 mg, about 75 mg to about 1000 mg, about 100 mg to about 1000 mg, about 125 mg to about 1000 mg, about 150 mg to about 1000 mg, about 175 mg to about 1000 mg, about 200 mg to about 1000 mg, about 225 mg to about 1000 mg, about 250 mg to about 1000 mg, about 300 mg to about 1000 mg, about 350 mg to about 1000 mg, about 400 mg to about 1000 mg, about 450 mg to about 1000 mg, about 500 mg to about 1000 mg, about 550 mg to about 1000 mg, about 600 mg to about 1000 mg, about 650 mg to about 1000 mg, about 700 mg to about 1000 mg, about 750 mg to about 1000 mg, about 800 mg to about 1000 mg, about 850 mg to about 1000 mg, about 900 mg to about 1000 mg, about 950 mg to about 1000 mg, about 1 mg to about 750 mg, about 2 mg to about 750 mg, about 5 mg to about 750 mg, about 10 mg to about 750 mg, about 25 mg to about 750 mg, about 50 mg to about 750 mg, about 75 mg to about 750 mg, about 100 mg to about 750 mg, about 125 mg to about 750 mg, about 150 mg to about 750 mg, about 175 mg to about 750 mg, about 200 mg to about 750 mg, about 225 mg to about 750 mg, about 250 mg to about 750 mg, about 300 mg to about 750 mg, about 350 mg to about 750 mg, about 400 mg to about 750 mg, about 450 mg to about 750 mg, about 500 mg to about 750 mg, about 550 mg to about 750 mg, about 600 mg to about 750 mg, about 650 mg to about 750 mg, about 700 mg to about 750 mg, about 1 mg to about 500 mg, about 2 mg to about 500 mg, about 5 mg to about 500 mg, about 10 mg to about 500 mg, about 25 mg to about 500 mg, about 50 mg to about 500 mg, about 75 mg to about 500 mg, about 100 mg to about 500 mg, about 125 mg to about 500 mg, about 150 mg to about 500 mg, about 175 mg to about 500 mg, about 200 mg to about 500 mg, about 225 mg to about 500 mg, about 250 mg to about 500 mg, about 300 mg to about 500 mg, about 350 mg to about 500 mg, about 400 mg to about 500 mg, about 450 mg to about 500 mg, about 1 mg to about 400 mg, about 2 mg to about 400 mg, about 5 mg to about 400 mg, about 10 mg to about 400 mg, about 25 mg to about 400 mg, about 50 mg to about 400 mg, about 75 mg to about 400 mg, about 100 mg to about 400 mg, about 125 mg to about 400 mg, about 150 mg to about 400 mg, about 175 mg to about 400 mg, about 200 mg to about 400 mg, about 225 mg to about 400 mg, about 250 mg to about 400 mg, about 300 mg to about 400 mg, about 350 mg to about 400 mg, about 1 mg to about 300 mg, about 2 mg to about 300 mg, about 5 mg to about 300 mg, about 10 mg to about 300 mg, about 25 mg to about 300 mg, about 50 mg to about 300 mg, about 75 mg to about 300 mg, about 100 mg to about 300 mg, about 125 mg to about 300 mg, about 150 mg to about 300 mg, about 175 mg to about 300 mg, about 200 mg to about 300 mg, about 225 mg to about 300 mg, about 250 mg to about 300 mg, about 1 mg to about 250 mg, about 2 mg to about 250 mg, about 5 mg to about 250 mg, about 10 mg to about 250 mg, about 25 mg to about 250 mg, about 50 mg to about 250 mg, about 75 mg to about 250 mg, about 100 mg to about 250 mg, about 125 mg to about 250 mg, about 150 mg to about 250 mg, about 175 mg to about 250 mg, about 200 mg to about 250 mg, about 225 mg to about 250 mg, about 1 mg to about 225 mg, about 2 mg to about 225 mg, about 5 mg to about 225 mg, about 10 mg to about 225 mg, about 25 mg to about 225 mg, about 50 mg to about 225 mg, about 75 mg to about 225 mg, about 100 mg to about 225 mg, about 125 mg to about 225 mg, about 150 mg to about 225 mg, about 175 mg to about 225 mg, about 200 mg to about 225 mg, about 1 mg to about 200 mg, about 2 mg to about 200 mg, about 5 mg to about 200 mg, about 10 mg to about 200 mg, about 25 mg to about 200 mg, about 50 mg to about 200 mg, about 75 mg to about 200 mg, about 100 mg to about 200 mg, about 125 mg to about 200 mg, about 150 mg to about 200 mg, about 175 mg to about 200 mg, about 180 mg to about 200 mg, about 1 mg to about 175 mg, about 2 mg to about 175 mg, about 5 mg to about 175 mg, about 10 mg to about 175 mg, about 25 mg to about 175 mg, about 50 mg to about 175 mg, about 75 mg to about 175 mg, about 100 mg to about 175 mg, about 125 mg to about 175 mg, about 150 mg to about 175 mg, about 1 mg to about 150 mg, about 2 mg to about 150 mg, about 5 mg to about 150 mg, about 10 mg to about 150 mg, about 25 mg to about 150 mg, about 50 mg to about 150 mg, about 75 mg to about 150 mg, about 100 mg to about 150 mg, about 125 mg to about 150 mg, about 1 mg to about 125 mg, about 2 mg to about 125 mg, about 5 mg to about 125 mg, about 10 mg to about 125 mg, about 25 mg to about 125 mg, about 50 mg to about 125 mg, about 75 mg to about 125 mg, about 100 mg to about 125 mg, about 1 mg to about 100 mg, about 2 mg to about 100 mg, about 5 mg to about 100 mg, about 10 mg to about 100 mg, about 25 mg to about 100 mg, about 50 mg to about 100 mg, about 60 mg to about 100 mg, or about 75 mg to about 100 mg.

In some embodiments, exemplary doses of the compound of Formula (I) or of Formula (II), or a pharmaceutically acceptable salt or co-crystal thereof, for a human subject may be about 1 mg, about 2 mg, about 5 mg, about 10 mg, about 15 mg, about 20 mg, about 25 mg, about 30 mg, about 35 mg, about 40 mg, about 45 mg, about 50 mg, about 60 mg, about 65 mg, about 70 mg, about 75 mg, about 100 mg, about 125 mg, about 150 mg, about 175 mg, about 180 mg, about 190 mg, about 200 mg, about 225 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1200 mg, about 1400 mg, about 1600 mg, about 1800 mg, about 2000 mg, about 2200 mg, about 2400 mg, about 2600 mg, about 2800 mg, about 3000 mg, about 3200 mg, about 3400 mg, about 3600 mg, about 3800 mg, about 4000 mg, about 4200 mg, about 4400 mg, about 4600 mg, about 4800 mg, or about 5000 mg.

In other embodiments, the methods provided comprise continuing to treat the subject (e.g., a human) by administering the doses of the compound of Formula (I) or of Formula (II), or a pharmaceutically acceptable salt or co-crystal thereof, at which clinical efficacy is achieved or reducing the doses by increments to a level at which efficacy can be maintained. In some embodiments, the methods provided comprise administering to the subject (e.g., a human in need thereof) an initial daily dose of 50 mg to about 500 mg or the compound of Formula (I) or of Formula (II), or a pharmaceutically acceptable salt or co-crystal thereof, or in an alternative embodiment 100 mg to 1000 mg of the compound of Formula (I) or of Formula (II), or a pharmaceutically acceptable salt or co-crystal thereof, and administering subsequent daily doses of the compound of Formula (I) or of Formula (II), or a pharmaceutically acceptable salt or co-crystal thereof, over at least 6 days, wherein each subsequent daily dose is increased by 25 mg to 300 mg, or by 50 mg to about 400 mg. Thus, it should also be understood that the dose of the compound of Formula (I) or of Formula (II), or a pharmaceutically acceptable salt or co-crystal thereof, may be increased by increments until clinical efficacy is achieved. Increments of about 10 mg, about 25 mg, about 50 mg, about 100 mg, or about 125 mg, or about 150 mg, or about 200 mg, or about 250 mg, or about 300 mg can be used to increase the dose. The dose can be increased daily, every other day, two, three, four, five or six times per week, or once per week. Initial doses of a compound of Formula (I) or of Formula (II), or a pharmaceutically acceptable salt or co-crystal thereof, may be selected from 250 mg, 300 mg, 350 mg, 400 mg, 450 mg, or 500 mg, each administered once, twice, or three times daily.

The frequency of dosing will depend on the pharmacokinetic parameters of the compound administered, the route of administration, and the particular disease treated. The dose and frequency of dosing may also depend on pharmacokinetic and pharmacodynamic, as well as toxicity and therapeutic efficiency data. For example, pharmacokinetic and pharmacodynamic information about the compound of Formula (I) or of Formula (II), or a pharmaceutically acceptable salt or co-crystal thereof, can be collected through preclinical in vitro and in vivo studies, later confirmed in humans during the course of clinical trials. Thus, for the compound of Formula (I) or of Formula (II), or a pharmaceutically acceptable salt or co-crystal thereof, used in the methods provided herein, a therapeutically effective dose can be estimated initially from biochemical and/or cell-based assays. Then, dosage can be formulated in animal models to achieve a desirable circulating concentration range that modulates Syk expression or activity. As human studies are conducted further information will emerge regarding the appropriate dosage levels and duration of treatment for various diseases and conditions.

Toxicity and therapeutic efficacy of the compound of Formula (I) or of Formula (II), or a pharmaceutically acceptable salt or co-crystal thereof, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the “therapeutic index”, which typically is expressed as the ratio LD₅₀/ED₅₀. Compounds that exhibit large therapeutic indices, i.e., the toxic dose is substantially higher than the effective dose, are preferred. The data obtained from such cell culture assays and additional animal studies can be used in formulating a range of dosage for human use. The doses of such compounds lies preferably within a range of circulating concentrations that include the ED₅₀ with little or no toxicity.

Compositions (including, for example, formulations and unit dosages) comprising a compound of Formula (I) or Formula (II), or a pharmaceutically acceptable salt or co-crystal thereof, can be prepared and placed in an appropriate container, and labeled for treatment of an indicated condition. Accordingly, provided is also an article of manufacture, such as a container comprising a unit dosage form of a compound of Formula (I) or Formula (II), or a pharmaceutically acceptable salt or co-crystal thereof, and a label containing instructions for use of the compounds. In some embodiments, the article of manufacture is a container comprising a unit dosage form of a compound of Formula (I) or Formula (II), or a pharmaceutically acceptable salt or co-crystal thereof, and at least one pharmaceutically acceptable vehicle. The article of manufacture may be a bottle, vial, ampoule, single-use disposable applicator, or the like, containing the pharmaceutical composition provided in the present disclosure. The container may be formed from a variety of materials, such as glass or plastic and in one aspect also contains a label on, or associated with, the container which indicates directions for use in the treatment of cancer or inflammatory conditions. It should be understood that the active ingredient may be packaged in any material capable of improving chemical and physical stability, such as an aluminum foil bag. In some embodiments, diseases or conditions indicated on the label can include, for example, treatment of cancer.

Another embodiment provides a pharmaceutical kit for the treatment of GVHD in a human, including the treatment of aGVHD and/or cGVHD, the kit comprising a pharmaceutically effective amount of a compound of Formula (I) or of Formula (II), or a pharmaceutically acceptable salt, co-crystal, ester, solvate, hydrate, isomer, tautomer, isotope, polymorph, or prodrug thereof, and instructions for use of the compound of Formula (I) or Formula (II) in the treatment of GVHD, including aGVHD and/or cGVHD. For example, a kit can comprise one or more unit dosage forms of a compound of Formula (I) or Formula (II), or a pharmaceutically acceptable salt or co-crystal thereof, and a package insert containing instructions for use of the composition in treatment of GVHD including aGVHD and/or cGVHD. In some embodiments, the kit comprises one or more unit dosage forms of a compound of Formula (I) or Formula (II), or a pharmaceutically acceptable salt or co-crystal thereof, and at least one pharmaceutically acceptable vehicle, and instructions for their use. In other embodiments, the kit comprises one or more unit dosage forms of a compound of Formula (I) or Formula (II), or a pharmaceutically acceptable salt or co-crystal thereof, at least one unit dosage form of another pharmaceutical agent useful in treating GVHD, such as those described herein, and instructions for their use.

### Synthesis

It will be understood that the compounds of Formula (I) or of Formula (II), or a pharmaceutically acceptable salt or co-crystal thereof, may be prepared by methods known in the art. For example, the compound of Formula (I), or a pharmaceutically acceptable salt or co-crystal thereof, and pharmaceutical formulations comprising it may be prepared by methods disclosed in U.S. Pat. Nos. 8,748,607 and 8,450,321, J. Med Chem., Vol. 57, Issue 9, pp. 3856-3873, US 2015/0038504, and US 2015/0038505.

The compounds of the disclosure may be prepared using methods disclosed herein and routine modifications thereof which will be apparent given the disclosure herein and methods well known in the art. Conventional and well-known synthetic methods may be used in addition to the teachings herein. The synthesis of compounds of Formula (II), or a pharmaceutically acceptable salt or co-crystal thereof, may be accomplished as described in the following examples. If available, reagents may be purchased commercially, e.g. from Sigma Aldrich or other chemical suppliers.

General Syntheses

Typical embodiments of the compounds of Formula (II), or a pharmaceutically acceptable salt or co-crystal thereof, in accordance with the present disclosure may be synthesized using the general reaction schemes described below. It will be apparent given the description herein that the general schemes may be altered by substitution of the starting materials with other materials having similar structures to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments of the present disclosure, inspection of the structure of the compound to be synthesized will provide the identity of each substituent group. The identity of the final product will generally render apparent the identity of the necessary starting materials by a simple process of inspection, given the examples herein.

Synthetic Reaction Parameters

The compounds of Formulas (I) and (II), or a pharmaceutically acceptable salt or co-crystal thereof, can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain a chiral center. Accordingly, if desired, such compounds can be prepared or isolated as pure stereoisomers, i.e., as individual enantiomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

The terms “solvent,” “inert organic solvent” or “inert solvent” refer to a solvent inert under the conditions of the reaction being described in conjunction therewith (including, for example, benzene, toluene, acetonitrile, tetrahydrofuran (“THF”), dimethylformamide (“DMF”), chloroform, methylene chloride (or dichloromethane), diethyl ether, methanol, pyridine and the like). Unless specified to the contrary, the solvents used in the reactions of the present disclosure are inert organic solvents, and the reactions are carried out under an inert gas, preferably nitrogen.

The term “q.s.” means adding a quantity sufficient to achieve a stated function, e.g., to bring a solution to the desired volume (i.e., 100%).

The following examples are included to demonstrate embodiments of the disclosure concerning preparation and testing of compounds of Formula (II), or a pharmaceutically acceptable salt or co-crystal thereof. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

List of abbreviations and acronyms.

| Abbreviation | Meaning |
|---|---|
| ° C. | Degree Celsius |
| anal | Analytical |
| ATP | Adenosine-5′-triphosphate |
| ATX II | Anemonia sulcata toxin |
| AcOH | Acetic acid |
| ACN | Acetonitrile |
| CAN | Ceric ammonium nitrate |
| CDI | 1,1′-carbonyldiimidazole |
| CHO | Chinese hamster ovary |
| conc. | Concentrated |
| d | Doublet |
| DABCO | 1,4-Diazabicyclo[2.2.2]octane |
| DAST | (Diethylamino)sulfur trifluoride |
| dd | Doublet of doublets |
| DCE | 1,2-dichloroethane |
| DCM | Dichloromethane |
| DEAD | Diethyl azodicarboxylate |
| DIPEA | N,N-diisopropylethylamine |
| DMAP | 4-dimethylaminopyridine |
| DME | 1,2-dimethoxyethane |
| DMF | Dimethylformamide |
| DMSO | Dimethylsulfoxide |
| dppf | 1,1′-Bis(diphenylphosphino)ferrocene |
| EA | Ethyl alcohol |
| ECF | Extracellular fluid |
| EDTA | Ethylenediaminetetraacetic acid |
| EGTA | Ethylene glycol tetraacetic acid |
| equiv/eq | Equivalents |
| ESI | Electrospray ionization |
| Ac | Acetate |
| Et | Ethyl |
| EtOAc | Ethyl Acetate |
| g | Grams |
| HEPES | (4-(2-Hydroxyethyl)-1-piperazineethanesulfonic acid) |
| HATU | 2-(7-Aza-1H-Benzotriazole-1-yl)-1,1,3,3- |
| | tetramethyluronium hexafluorophosphate |
| hERG | human Ether-a-go-go Related Gene |
| HMDS | hexamethyldisilazane(azide) |
| HPLC | High-performance liquid chromatography |
| h | Hours |
| Hz | Hertz |
| IPA | Isopropyl alcohol |
| IC₅₀ | The half maximal inhibitory concentration |
| IMR-32 | Human neuroblastoma cell line |
| J | Coupling constant |
| Kg | Kilogram |
| kHz | Kilohertz |
| LAH | Lithium ammonium hydride |
| LCMS/LC-MS | Liquid chromatography-mass spectrometry |
| M | Molar |
| m | multiplet |
| m/z | mass-to-charge ratio |
| M+ | Mass peak |
| M + H | Mass peak plus hydrogen |
| mCPBA | 3-chloroperoxybenzoic acid |
| Me | Methyl |
| MeOH | Methanol |
| mg | Milligram |
| MHz | Megahertz |
| min/m | Minute |
| ml/mL | Milliliter |
| mM | Millimolar |
| mmol | Millimole |
| nmol | Nanomole |
| mOsmol | Milliosmole |
| MRM | Magnetic Resonance Microscopy |
| MS | Mass spectroscopy |
| ms | Millisecond |
| mV | Millivolt |
| mw | Microwave |
| N | Normal |
| mol | Mole |
| NMP | N-methylpyrrolidinone |
| NMR | Nuclear magnetic resonance |
| pA | Picoamps |
| Ph | Phenyl |
| ppm | Parts per million |
| prep | Preparative |
| q.s. | Quantity sufficient to achieve a stated function |
| Rf | Retention factor |
| RP | Reverse phase |
| RT/rt | Room temperature |
| s | Second |
| s | Singlet |
| SEM | 2-(Trimethylsilyl)ethoxymethyl |
| t | Triplet |
| TB | Tonic Block |
| TEA | Triethylamine |
| TFA | Trifluoroacetic acid |
| THF | Tetrahydrofuran |
| TLC | Thin layer chromatography |
| TMS | trimethylsilyl |
| TTX | Tetrodotoxin |
| UDB | Use Dependent Block |
| WT | Wild type |
| δ | Chemical shift |
| μg | Microgram |
| μL/μl | Microliter |
| μM | Micromolar |
| μm | Micrometer |
| μmol | Micromole |

### EXAMPLES

### Preparation of Common Intermediates

### Intermediate 1.01. Preparation of tert-Butyl (6-bromoimidazo[1,2-a]pyrazin-8-yl)(4-(4-(oxetan-3-yl)piperazin-1-yl)phenyl)carbamate IV and tert-butyl 4-(4-(oxetan-3-yl)piperazin-1-yl)phenyl(6-(tributylstannyl)imidazo[1,2-a]pyrazin-8-yl)carbamate V

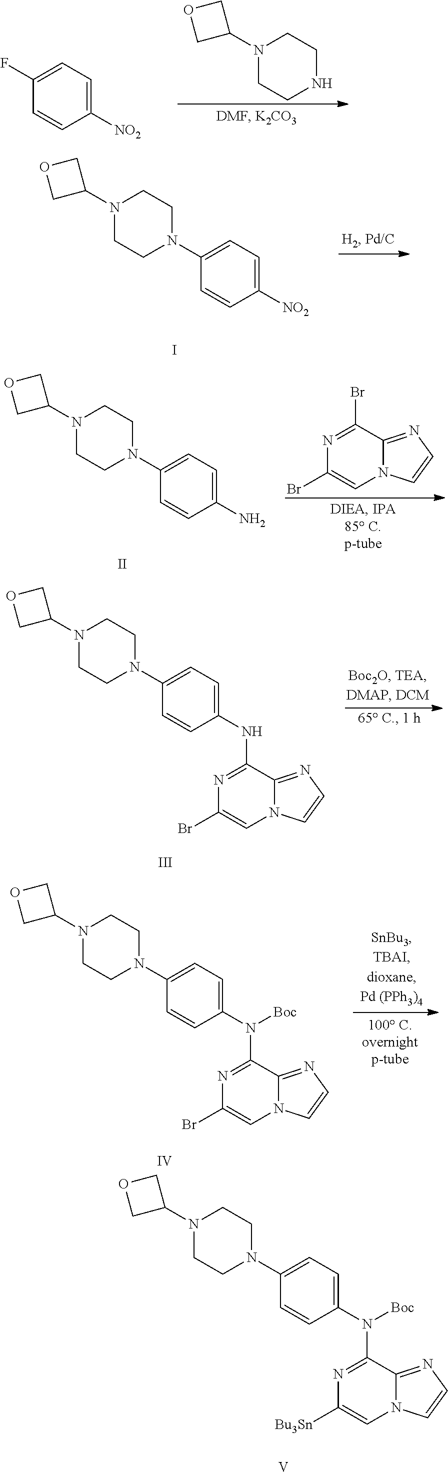

1-(4-Nitrophenyl)-4-(oxetan-3-yl)piperazine I: In a 500 mL round bottom flask, 1-(oxetan-3-yl)piperazine (3.02 g, 21.26 mmoles), potassium carbonate (5.87 g, 42.52 mmoles), 1-fluoro-4-nitrobenzene (3.00 g, 21.26 mmoles) was combined in acetonitrile (33 mL) and stirred under nitrogen overnight at 100° C. The mixture was diluted with water (100 mL) and extracted with DCM (100 mL×3), dried over anhydrous sodium carbonate, filtered and the filtrate was concentrated. The residue was dissolved in minimal DCM using a sonicator and crashed out with hexane. The precipitate was filtered, washed with hexane and dried to provide the title compound I.

4-(4-(Oxetan-3-yl)piperazin-1-yl)aniline II: In a hydrogenation vessel, 1-(4-nitrophenyl)-4-(oxetan-3-yl)piperazine I (4.70 g, 17.85 mmoles) was dissolved as much as possible in MeOH (26 mL) and DCM (5 mL). Pd/C (10%) (2.85 g, 2.68 mmoles) was added and the reaction was stored under nitrogen. The reaction was shaken on the Parr hydrogenator at 45 PSI. After 15 minutes, the reaction was fully recharged to 45 PSI and shaken for an additional hour. The material was filtered over celite, washed with 25% MeOH/DCM and concentrated to provide the title compound II.

6-Bromo-N-(4-(4-(oxetan-3-yl)piperazin-1-yl)phenyl)imidazo[1,2-a]pyrazin-8-amine III: To 4-(4-(oxetan-3-yl)piperazin-1-yl)aniline II (2.00 g, 8.57 mmoles), Hunig's base (3.29 mL) and 6,8-dibromoimidazo[1,2-a]pyrazine (2.37 g, 8.57 mmoles) was added in DMF (43 mL). The reaction was stirred at 85° C. in a pressure tube for overnight. The material was quenched with saturated sodium bicarbonate, extracted with DCM (120 mL×3) and the organic layers were combined and washed with water (120 mL×3), dried over anhydrous sodium carbonate and concentrated. The crude material was purified using a 120 g Isco column and eluted off using a stepwise gradient of 0-60% (10% MeOH/DCM). The desired fractions were combined and concentrated to provide the title compound III.

tert-Butyl (6-bromoimidazo[1,2-a]pyrazin-8-yl)(4-(4-(oxetan-3-yl)piperazin-1-yl)phenyl)carbamate IV: 6-bromo-N-(4-(4-(oxetan-3-yl)piperazin-1-yl)phenyl)imidazo[1,2-a]pyrazin-8-amine III (1000 mg, 2.33 mmol), di-tert-butyl dicarbonate (1016.72 mg, 4.66 mmol) and N,N-dimethylpyridin-4-amine (21.34 mg, 0.17 mmol) were stirred in DCM (1.01 ml) and refluxed at 65° C. for 3 h. The reaction was diluted with 100 mL of DCM, washed with H2O (×3), dried, filtered and concentrated. The crude material was dissolved in minimal DCM, loaded onto a preloaded silica loader and eluted off a 40 g column using 0-30% MeOH/DCM over 20 column volumes. The desired fractions were combined and concentrated to provide the title compound. This compound is used in Example 2.

tert-Butyl 4-(4-(oxetan-3-yl)piperazin-1-yl)phenyl(6-(tributylstannyl)imidazo[1,2-a]pyrazin-8-yl)carbamate V: In a 350 mL p-tube, tert-butyl 6-bromoimidazo[1,2-a]pyrazin-8-yl(4-(4-(oxetan-3-yl)piperazin-1-yl)phenyl)carbamate IV (8150 mg, 15.39 mmol), 1,1,1,2,2,2-hexabutyldistannane (11.67 ml, 23.09 mmol), tetrakis(triphenylphosphine)palladium (889.43 mg, 0.77 mmol), and tetrabutylammonium iodide (5686.03 mg, 15.39 mmol) were combined in dioxane (62 ml) and heated to 110° C. overnight. According to LCMS, no starting material remained. The reaction was absorbed onto celite and eluted off a 160 g alumina column using a 0-10-20-30-100% (50% EtOAc/Hex-Hex) gradient holding at 50% for 10-15 column volumes over 50-60 column volumes to provide the title compound V. This compound is used in Examples 1 and 2.

### Intermediate 1.02. Preparation tert-butyl (6-bromo-5-methylimidazo[1,2-a]pyrazin-8-yl)(4-(4-(oxetan-3-yl)piperazin-1-yl)phenyl)carbamate X

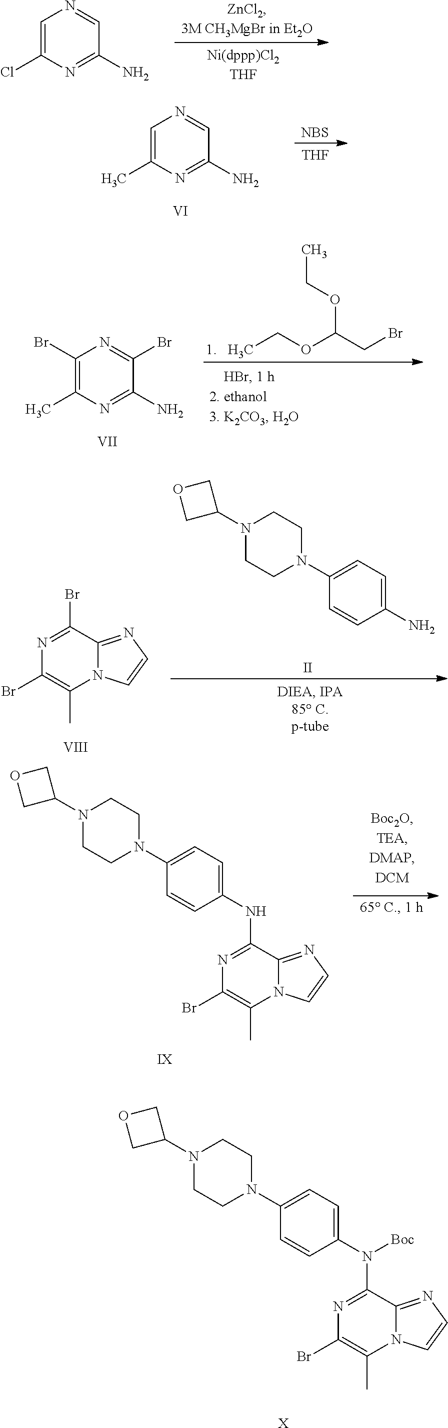

6-Methylpyrazin-2-amine VI: To a solution of anhydrous zinc(II) chloride (26.3 g, 193 mmol) in THF (150 mL) at 0° C., was added 3 M methyl magnesium bromide in diethyl ether (129 mL) drop wise over a period of 1 h. [1,3-bis(diphenylphosphino)propane] nickel(II) chloride (2.08 g, 3.85 mmol) was then added and the mixture allowed to warm to room temperature. To the above mixture, a solution of 6-chloro-2-aminopyrazine (5.00 g, 38.6 mmol) in anhydrous THF (25 mL) was added and the reaction stirred, under a nitrogen atmosphere, at reflux for 6 h. After this time, the mixture was cooled to room temperature, then to 0° C. and carefully quenched with saturated aqueous ammonium chloride (50 mL). The organic layer was separated and dried over sodium sulfate. The drying agent was filtered and the filtrate concentrated under reduced pressure to provide crude 6-methylpyrazin-2-amine VI, which was used in the next step without purification: ¹H NMR (400 MHz, CDCl₃) δ: 7.63 (s, 1H), 7.53 (s, 1H), 4.96 (bs, 2H), 2.16 (s, 3H).

3,5-Dibromo-6-methylpyrazin-2-amine VII: To a solution of 6-methylpyrazin-2-amine VI (2.00 g, 18.3 mmol) in THF (40 mL) at 10° C., was added N-bromosuccinimide (6.70 g, 37.6 mmol) portion wise over 15 min and the mixture allowed to warm to room temperature while stirring. After 2 h, the reaction was concentrated under reduced pressure and the resulting residue was purified by column chromatography (silica, gradient, hexanes to EtOAc) to provide 3,5-dibromo-6-methylpyrazin-2-amine VII: ¹H NMR (400 MHz, CDCl₃) δ: 4.93 (bs, 2H), 2.38 (s, 3H).

6,8-Dibromo-5-methylimidazo[1,2-a]pyrazine VIII: A mixture of 2-bromo-1,1-diethoxyethane (3.21 mL, 20.7 mmol) and 48% aqueous hydrobromic acid (1.0 mL) was stirred at reflux for 2 h. The reaction was then cooled to room temperature and treated with sodium bicarbonate until gas evolution ceased. The mixture was filtered and the filtrate diluted with ethanol (15 mL). To this mixture, 3,5-dibromo-6-methylpyrazin-2-amine VII (3.00 g, 11.2 mmol) was added and the reaction stirred at reflux for 16 h. After this time, the reaction was cooled to room temperature and concentrated under reduced pressure to a volume of approximately 10 mL. The suspension was filtered and the filter cake washed with cold ethanol (5 mL). The filter cake was then taken into water (50 mL) and the pH was adjusted to ˜8 with potassium carbonate. The resulting suspension was filtered and the filter cake dried to a constant weight under vacuum to provide 6,8-dibromo-5-methylimidazo[1,2-a]pyrazine VIII: ¹H NMR (400 MHz, CDCl₃) δ: 7.90 (s, 1H), 7.72 (s, 1H), 2.74 (s, 3H).

6-Bromo-5-methyl-N-(4-(4-(oxetan-3-yl)piperazin-1-yl)phenyl)imidazo[1,2-a]pyrazin-8-amine IX: The compound IX was prepared from 6,8-dibromo-5-methylimidazo[1,2-a]pyrazine VIII using the method as described for preparing 6-bromo-N-(4-(4-(oxetan-3-yl)piperazin-1-yl)phenyl)imidazo[1,2-a]pyrazin-8-amine III in Intermediate Example 1.01.

tert-Butyl (6-bromo-5-methylimidazo[1,2-a]pyrazin-8-yl)(4-(4-(oxetan-3-yl)piperazin-1-Aphenyl)carbamate X: The compound X was prepared from 6-bromo-5-methyl-N-(4-(4-(oxetan-3-yl)piperazin-1-yl)phenyl)imidazo[1,2-a]pyrazin-8-amine IX using the method as described for preparing tert-butyl (6-bromoimidazo[1,2-a]pyrazin-8-yl)(4-(4-(ocetan-3-yl)piperazin-1-yl)phenyl)carbamate IV in Intermediate Example 1.01. This compound is used in Example 4.

### Synthesis of Examples 1-7

### Example 1

### Preparation of 6-(6-amino-5-methylpyrazin-2-yl)-N-(4-(4-(oxetan-3-yl)piperazn-1-yl)phenyl)imidazo[1,2-a]pyrazin-8-amine (1)

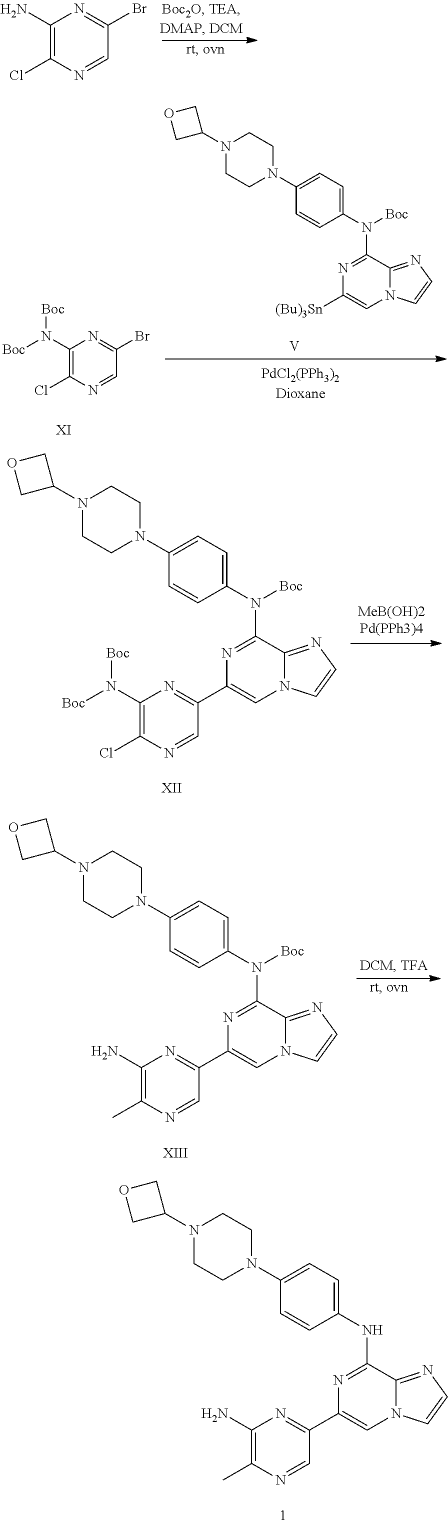

2-Bis(tert-butoxycarbonyl)amino-6-bromo-3-chloropyrazine XI: 6-Bromo-3-chloropyrazin-2-amine (2000 mg, 9.59 mmol) was dissolved in DCM (48 ml) followed by triethylamine (3.99 ml, 28.78 mmol), di-tert-butyl dicarbonate (4188.12 mg, 19.19 mmol), and N,N-dimethylpyridin-4-amine (87.91 mg, 0.72 mmol). The reaction was allowed to stir at room temperature for overnight. The crude material was washed with water, dried, filtered and concentrated. The crude material was dissolved in minimal DCM and loaded onto a 25 g prepacked silica loader and eluted off a 40 g column using 0-30% MeOH/DCM. The title compound XI was isolated and identified by LCMS and NMR. The product was a mix of mono and bis boc-protected material, mainly bis boc-protected as observed by NMR.

tert-Butyl tert-butoxycarbonyl(6-(8-((tert-butoxycarbonyl)(4-(4-(oxetan-3-yl)piperazin-1-yl)phenyl)amino)imidazo[1,2-a]pyrazin-6-yl)-3-chloropyrazin-2-yl)carbamate XII: tert-Butyl 4-(4-(Oxetan-3-yl)piperazin-1-yl)phenyl(6-(tributylstannyl)imidazo[1,2-a]pyrazin-8-yl)carbamate V (1000 mg, 1.4 mmol), 2-Bis(tert-butoxycarbonyl)amino-6-bromo-3-chloropyrazine XI (552 mg, 1.35 mmol), and PdCl₂(PPh₃)₂ (142.77 mg, 0.20 mmol), in 1,4-Dioxane (11.27 ml) was irridated in the microwave for 20 min at 140° C. The reaction was absorbed onto celite and eluted off a 40 g Gold Isco column using 0-10-100% (30% MeOH/DCM) over 20 column volumes. Fractions 34-39 were collected and concentrated. According to NMR, the title compound XII was identified and isolated.

tert-Butyl (6-(6-amino-5-methylpyrazin-2-yl)imidazo[1,2-a]pyrazin-8-yl)(4-(4-(oxetan-3-yl)piperazin-1-yl)phenyl)carbamate XIII: In a microwave vial, tert-butyl tert-butoxycarbonyl(6-(8-((tert-butoxycarbonyl)(4-(4-(oxetan-3-yl)piperazin-1-yl)phenyl)amino)imidazo[1,2-a]pyrazin-6-yl)-3-chloropyrazin-2-yl)carbamate XII (300 mg, 0.44 mmol), methylboronic acid (794.39 mg, 13.27 mmol), tetrakis(triphenylphosphine)palladium (51.12 mg, 0.04 mmol), and 2M Na₂CO₃ (0.44 ml) were combined in DME (1.77 ml) and irridated in the microwave for 20 min at 150° C. The reaction was worked up using 25% MeOH/DCM and water. The organic layers were combined, dried, filtered and concentrated. The crude material was loaded onto silica and eluted off a 40 g Gold column using 0-5-15-25-50% (30% MeOH/DCM) over 45 column volumes. The desired fractions were concentrated and provided tert-butyl (6-(6-amino-5-methylpyrazin-2-yl)imidazo[1,2-a]pyrazin-8-yl)(4-(4-(oxetan-3-yl)piperazin-1-yl)phenyl)carbamate XIII as the minor product and the desired final compound 1 as an inseparable mixture (208 mg total) and were taken in to the TFA reaction.

6-(6-Amino-5-methylpyrazin-2-yl)-N-(4-(4-(oxetan-3-yl)piperazin-1-yl)phenyl)imidazo[1,2-a]pyrazin-8-amine (1): To a solution of tert-butyl 6-(6-amino-5-methylpyrazin-2-yl)imidazo[1,2-a]pyrazin-8-yl(4-(4-(oxetan-3-yl)piperazin-1-yl)phenyl)carbamate XIII (48 mg, 0.09 mmol) and 6-(6-amino-5-methylpyrazin-2-yl)-N-(4-(4-(oxetan-3-yl)piperazin-1-yl)phenyl)imidazo[1,2-a]pyrazin-8-amine (1, 160 mg, 0.35 mmol) in DCM (2.5 ml) was added TFA (0.16 ml, 2.15 mmol). Additional TFA (0.48 ml, 6.5 mmol) was added to the reaction mixture to ensure reaction completion. The reaction was then cooled to 0° C. and quenched with sat. NaHCO₃, then extracted with DCM (5 ml×3), and the combined organic layers were washed with water (5 ml×2), brine (5 ml×1), dried (Na₂SO₄), and concentrated to give the crude product. The crude material was absorbed onto silica and eluted off a 24 g Gold Isco column using 0-15-25-40-100% (30% MeOH/DCM). The desired fractions were combined and concentrated to provide the desired compound. LCMS-ESI⁺ (m/z): [M+H]⁺: 458.22. ¹H NMR (300 MHz, d₆-DMSO) δ: 9.48 (s, 1H), 8.54 (s, 1H), 8.41 (s, 1H),8.11 (s, 1H), 7.95 (d, 2H), 7.6 (s,1H), 6.98 (d, 2H), 6.2 (s, 2H), 4.58-4.45 (dt, 4H), 3.3 (m, 1H), 3.14 (t, 4H), 2.50-2.4 (dt,4H), 2.33 (s, 1H). Alternatively, compound XII could be taken directly to this step and similarly de-protected to provide the 5-chloropyrazine substituted analog.

### Example 2

### Preparation of 6-(6-aminopyrazin-2-yl)-N-(4-(4-(oxetan-3-yl)piperazin-1-yl)phenyl)imidazo[1,2-a]pyrazin-8-amine (2)

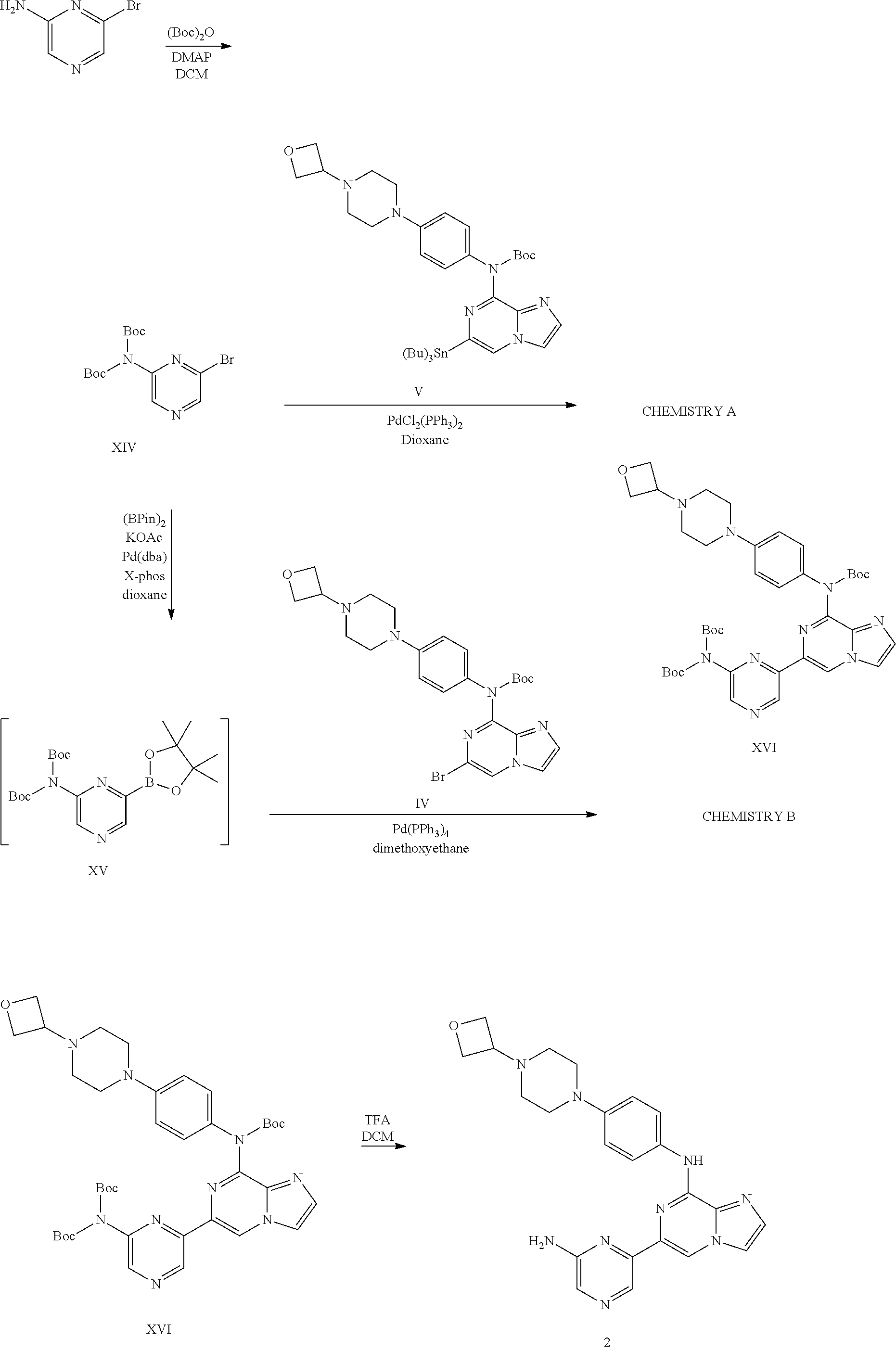

2-Bis(tert-butoxycarbonyl)amino-6-bromopyrazine XIV: To a mixture of 6-bromopyrazin-2-amine (5 g, 28.7 mmol) and di-tert-butyl dicarbonate (25.09 g, 114.94 mmol) was added DCM (10 ml) followed by DMAP (0.351 g, 29 mmol). The reaction was heated to 55° C. for 1 h, cooled to RT, the reaction was partitioned between water and DCM, purified on silica gel and concentrated to provide of 2-bis(tert-butoxycarbonyl)amino-6-bromopyrazine XIV. LCMS-ESI⁺ (m/z): [M+H]⁺: 374.14. ¹H NMR (DMSO) δ: 8.84(d, 2H), 1.39 (s, 18H).

tert-Butyl (6-(6-(bis(tert-butoxycarbonyl)amino)pyrazin-2-yl)imidazo[1,2-a]pyrazin-8-yl)(4-(4-(oxetan-3-yl)piperazin-1-yl)phenyl)carbamate XVI—CHEMISTRY A route: tert-Butyl 4-(4-(oxetan-3-yl)piperazin-1-yl)phenyl(6-(tributylstannypimidazo[1,2-a]pyrazin-8-yl)carbamate V (215 mg, 0.291 mmol), was combined with 2-bis(tert-butoxycarbonyl)amino-6-bromopyrazine XIV (217.58 mg, 0.581 mmol), bis(triphenylphosphine)palladium(II) dichloride(30.61 mg, 0.044 mmol) and 1,4-dioxane (5 ml). The reaction mixture was stirred in a microwave reactor at 120° C. for 30 min. The reaction mixture was quenched with saturated KF, extracted with EtOAc, purified on silica gel, eluted with EtOAc. The desired fractions were combined and concentrated to provide 100 mg (46% yield) of tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)phenyl)carbamate XVI. LCMS-ESI⁺ (m/z): [M+H]⁺: 744.4. ¹H NMR (300 MHz d₆-DMSO) δ: 9.37 (s, 1H), 9.18 (s, 1H), 8.77 (s, 1H), 8.33 (d, 1H), 7.87 (d, 1H), 7.28-7.25 (d, 2H), 6.92-6.89 (d, 2H), 4.55-4.41 (m, 4H), 3.4 (m,1H), 3.14-3.11 (m,4H), 2,37-2.34 (m, 4H), 1.37 (s, 18H), 1.3 (s, 9H).

tert-Butyl (6-(6-(bis(tert-butoxycarbonyl)amino)pyrazin-2-yl)imidazo[1,2-a]pyrazin-8-yl)(4-(4-(oxetan-3-yl)piperazin-1-yl)phenyl)carbamate XVI—CHEMISTRY B route: Step 1: To a dry 250 mL round-bottomed flask was added 2-bis(tert-butoxycarbonyl)amino-6-bromopyrazine XIV (1.0 g, 1.0 equiv, 2.67 mmol), KOAc (790 mg, 8.02 mmol, 3.0 equiv), 4,4,4′,4′,5,5,5′,5′-octamethyl-2,2′-bi(1,3,2-dioxaborolane) (750 mg, 2.94 mmol, 1.1 equiv), Pd(dba) (171 mg, 0.187 mmol, 0.07 equiv) and X-phos (128 mg, 0.267 mmol, 0.1 equiv) followed by 1,4-dioxane (25 mL) and the solution was sonicated for 5 min and then purged with N₂ gas for 5 min. The flask with contents was then placed under N₂ atmosphere and heated at 110° C. for 90 min. Once full conversion to the pinacolboronate was achieved by LCMS, the reaction was removed from heat and allowed to cool to RT. Once cool, the reaction contents were filtered through Celite and the filter cake was washed 3×20 mL EtOAc. The resultant solution was then concentrated down to a deep red-orange syrup providing N, N-BisBoc 6-(4,4,5,5-tetramethyl-1,3,2-dioxaborolan-2-yl)pyrazin-2-amine XV, which was used directly in the next step.

Step 2: The freshly formed N, N-BisBoc 6-(4,4,5,5-tetramethyl-1,3,2-dioxaborolan-2-yl)pyrazin-2-amine XV (2.67 mmol based on 100% conversion, 2.0 equiv based on bromide) was dissolved in 20 Ml of 1,2-dimethoxyethane and to that solution was added tert-butyl (6-bromoimidazo[1,2-a]pyrazin-8-yl)(4-(4-(oxetan-3-yl)piperazin-1-y1)phenyl)carbamate IV (707 mg, 1.34 mmol, 1.0 equiv), Na₂CO₃ (283 mg, 2.67 mmol, 2.0 equiv), Pd(PPh₃)₄ (155 mg, 0.134 mmol, 0.lequiv) and water (10 mL) and the solution was degassed for 5 min using N₂ gas. The reaction was then placed under N₂ atmosphere and heated at 110° C. for 90 min. LCMS showed complete consumption of the bromide starting material and the reaction was removed from heat and allowed to cool to RT. The reaction was diluted with 100 mL water and 100 mL 20% MeOH/DCM and the organic layer was recovered, extracted 1× sat. NaHCO₃, 1× sat brine and then dried over Na₂SO₄. The solution was then filtered and concentrated down to an orange-red solid. The sample was then slurried in warm MeOH, sonicated then filtered, washing 2×20 mL with cold MeOH and then the cream-colored solid was dried on hi-vacuum overnight to yield 905 mg of tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)phenyl)carbamate XVI.

6-(6-Aminopyrazin-2-yl)-N-(4-(4-(oxetan-3-yl)piperazin-1-yl)phenyl)imidazo[1,2-a]pyrazin-8-amine (2): To a solution of tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)phenyl)carbamate XVI (200 mg, 0.269 mmol) in DCM (2 ml) was added TFA (0.5 ml, 6.578 mmol). The reaction was stirred at rt for 16h, saturated sodium bicarbonate was added, extracted with EtOAC and purified on silica gel, eluted with 5% MeOH/EtOAc, 20% MeOH/EtOAc. The desired fractions were combined and concentrated to provide the title compound 2. LCMS-ESI⁺ (m/z): [M+H]⁺: 444.2. ¹H NMR (300 MHz d₆-DMSO) δ: 9.5 (s,1H), 8.588 (s, 1H), 8.47 (s, 1H), 8.12 (d, 1H), 7.95-7.92 (d, 2H), 7.88 (s, 1H), 7.62 (s, 1H), 6.99-6.96 (d, 2H), 6.46 (s, 2H), 4.57-4.53 (m, 2H), 4.48-4.44 (m, 2H), 3.43 (m, 1H), 3.15-3.12 (m, 4H), 2.41-2.38 (m, 4H).

### Example 2

### Alternate Synthesis

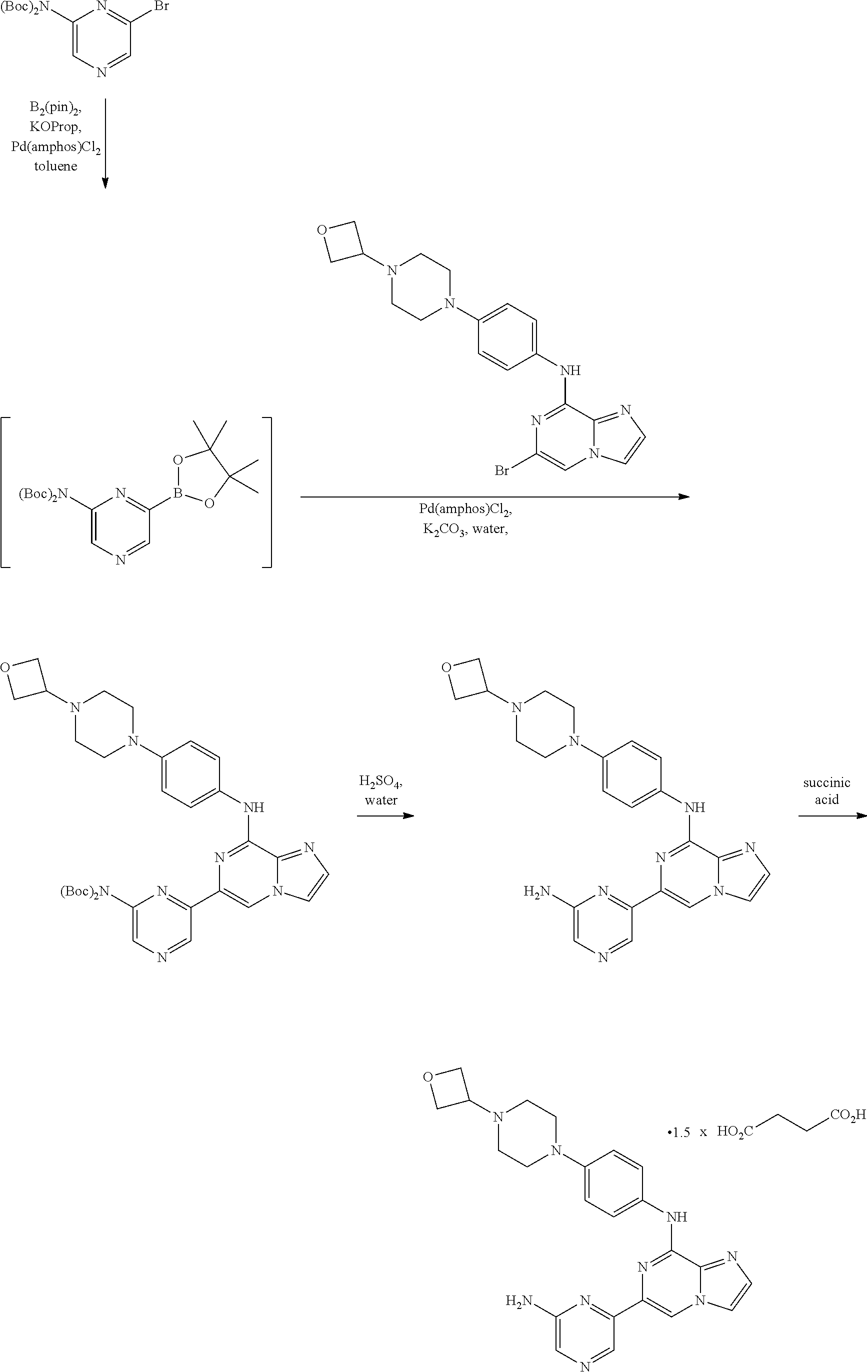

### Di-tert-butyl {6-[8-({4-[4-(oxetan-3-yl)piperazin-1-yl]phenyl}amino)imidazo[1,2-a]pyrazin-6-yl]pyrazin-2-yl}imidodicarbonate

To a 720 L reactor, was added di-tent-butyl (6-bromopyrazin-2-yl)imidodicarbonate (18.5 kg, 1.41 equiv, 49 mol), bis(pinacolato)diboron (13.8 kg, 1.56 equiv, 54 mol), potassium propionate (11.9 kg, 3.02 equiv, 106 mol), and bis(di-tert-butyl(4-dimethylaminophenyl) phosphine)dichloropalladium (1.07 kg, 0.0043 equiv, 1.5 mol), followed by degassed toluene (173 L). The mixture was degassed then heated at 65° C. until the reaction was deemed complete (0% tert-butyl 2-((6-bromopyrazin-2-yl)(tert-butoxycarbonyl)amino)-2-oxoacetate) by UPLC. Upon completion, the reaction was cooled to 23° C. Once cooled, 6-bromo-N-(4-(4-(oxetan-3-yl)piperazin-1-yl)phenyl)imidazo[1,2-a]pyrazin-8-amine (15.0 kg, 1.00 equiv, 35 mol) was added and the mixture was degassed. A degassed aqueous potassium carbonate solution prepared using water (54 L) and potassium carbonate (20.6 g, 4.26 equiv, 149 mol) was then added to the reaction mixture and the reactor contents was degassed. The reactor contents was heated at 65° C. until reaction was deemed complete (1% 6-bromo-N-(4-(4-(oxetan-3-yl)piperazin-1-yl)phenyl)imidazo[1,2-a]pyrazin-8-amine) by UPLC. Upon completion, the reaction was cooled to 24° C.

The cooled mixture was concentrated and then diluted with dichloromethane (300 L), transferred to a 1900 L reactor and rinsed forward with dichloromethane (57 L). N-acetyl-L-cysteine (3.8 kg) was charged and the mixture was agitated for 15 h. Water (135 L) was then added and the mixture was filtered and rinsed forward with dichloromethane (68 L). The organic layer was recovered and washed with a brine solution prepared using water (68 L) and sodium chloride (7.5 kg).

The resultant organic layer was polish filtered then concentrated and tert-butyl methyl ether (89.9 kg) was slowly charged keeping the temperature at 31° C. The contents was cooled to 0° C. and aged, then filtered and rinsed with tert-butyl methyl ether (32.7 kg) and dried at 40° C. to give 17.2 kg of di-tent-butyl {6-[8-({4-4-(oxetan-3-yl)piperazin-1-yl]phenyl}amino)imidazo[1,2-a]pyrazin-6-yl]pyrazin-2-yl}imidodicarbonate.

LCMS-ESI⁺ (m/z): [M+H]⁺: 644.3. ¹H NMR (400 MHz, CDCl₃) δ: 9.43 (s, 1H), 8.58 (s, 1H), 8.53 (s, 1H), 8.02 (s, 1H), 7.84 (m, 2H), 7.63 (d, 1H), 7.61 (d, 1H), 7.04 (m, 2H), 4.71 (m,4H), 3.59 (m,1H), 3.27 (m, 4H), 2.55 (m, 4H), 1.46 (s, 18H).

### 6-(6-Aminopyrazin-2-yl)-N-(4-(4-(oxetan-3-yl)piperazin-1-yl)phenyl)imidazo[1,2-a]pyrazin-8-amine succinate (Example 2)

To a slurry of di-tert-butyl }6-[8-(}4-[4-(oxetan-3-yl)piperazin-1-yl]phenyl}amino)imidazo[1,2-a]pyrazin-6-yl]pyrazin-2-yl}imidodicarbonate (225 g, 0.35 mol, 1 mol eq.) in water (12 parts) was added a solution of sulfuric acid (3.1 parts, 6.99 mol, 20 mol eq.) in water (5 parts). The reaction was heated to ca. 40° C. and stirred at this temperature for ca. 4 h at which point the reaction is deemed complete. The reaction mixture was cooled to ca. 22° C., acetone (3 parts) was charged and a solution of sodium carbonate (4.1 parts, 8.75 mol, 25.0 mol eq.) in water (15 parts) was added. The resulting slurry was filtered and the wet cake was washed with water in portions (4×1 parts), then with tert-butyl methyl ether (4 parts). The wet cake (Example 2 free base) was dried at ca. 60° C. To the slurry of dry Example 2 free base in 2-propanol (2.3 parts) was added a solution of succinic acid (Based on the isolated Example 2 free base: 0.43 parts, 1.6 mol eq.) in 2-propanol (15 parts). The resulting slurry was heated to ca. 40° C. and stirred at this temperature for ca. 2 h and then cooled to ca. 22° C., followed by a stir period of ca. 16 h. The slurry was filtered at ca. 22° C. and the wet cake was washed with 2-propanol (5 parts) and dried at ca. 60° C. to afford the product.

LCMS-ESI⁺ (m/z): [M+H]⁺: 620.65. ¹H NMR (400 MHz d₆-DMSO) δ: 12.2 (broad s,1.5H), 9.58 (s, 1H), 8.63 (s, 1H), 8.50 (s, 1H), 8.15 (s, 1H), 7.95 (d, 2H), 7.90 (s, 1H), 7.64 (s, 1H), 7.00 (d, 2H), 6.50 (s, 2H), 4.52 (dd, 4H), 3.45 (m, 1H), 3.19 (m, 4H), 2.40 (m, 10H).

### Example 3

### Preparation of (R)-(4-(4-((6-(6-aminopyrazin-2-yl)imidazo[1,2-a]pyrazin-8-yl)amino)phenyl)morpholin-2-yl)methanol (3)

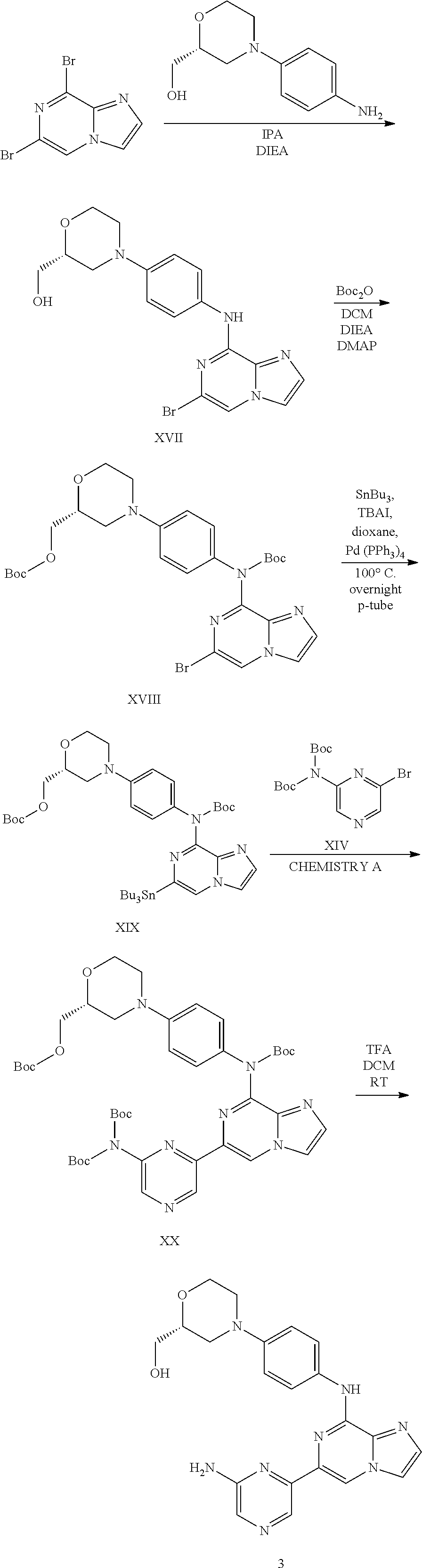

(R)-(4-(4-((6-Bromoimidazo[1,2-a]pyrazin-8-yl)amino)phenyl)morpholin-2-yl)methanol XVII: In a 250 mL round bottom flask equipped with a condenser was placed 6,8-dibromoimidazo[1,2-a]pyrazine (2000 mg, 7.22 mmol) and added 30 mL isopropanol followed by N,N-diisopropylethylamine (2.52 ml, 14.44 mmol) and (R)-(4-(4-aminophenyl)morpholin-2-yl)methanol (1504.12 mg, 7.22 mmol). The reaction was heated to reflux (oil bath 95° C.) overnight. The reaction was cooled and precipitates were collected by filtration and washed with isopropanol followed by hexanes to give the desired compound XVII.

(R)-tert-Butyl (6-bromoimidazo[1,2-a]pyrazin-8-yl)(4-(2-(((tert-butoxycarbonyl)oxy)methyl)morpholino)phenyl)carbamate XVIII: In a 250 mL round bottom flask was placed (R)-(4-(4-((6-bromoimidazo[1,2-a]pyrazin-8-yl)amino)phenyl)morpholin-2-yl)methanol XVII (2.80g, 6.9 mmol) and added DCM followed by triethylamine (2.9 mL, 2.1 g, 20.8 mmol), DMAP (63 g, 0.52 mmol) and di-tert-butyl dicarbonate (3.8 g, 17.3 mmol). The reaction was stirred overnight then diluted with DCM and water, separated, washed with brine, dried over Na₂SO₄, filtered and concentrated under reduced pressure. The crude material was purified by chromatography: ISCO 40 g silica with 25 g silica loader, eluting with 0-100% EtOAc/hexanes to give compound XVIII.

(R)-tert-Butyl (4-(2-(((tert-butoxycarbonyl)oxy)methyl)morpholino)phenyl)(6-(tributylstannyl)imidazo[1,2-a]pyrazin-8-yl)carbamate XIX: (R)-tert-Butyl (6-bromoimidazo[1,2-a]pyrazin-8-yl)(4-(2-(((tert-butoxycarbonyl)oxy)methyl)morpholino)phenyl)carbamate XVIII was reacted according to the analogous method of Example Intermediate 1.01 to provide (R)-tert-butyl (4-(2-(((tert-butoxycarbonyl)oxy)methyl)morpholino)phenyl)(6-(tributylstannyl)imidazo[1,2-a]pyrazin-8-yl)carbamate XIX.

(R)-tert-Butyl (6-(6-(bis(tert-butoxycarbonyl)amino)pyrazin-2-yl)imidazo[1,2-a]pyrazin-8-yl)(4-(2-(((tert-butoxycarbonyl)oxy)methyl)morpholino)phenyl)carbamate XX: (R)-tert-Butyl (4-(2-(((tert-butoxycarbonyl)oxy)methyl)morpholino)phenyl)(6-(tributylstannyl)imidazo[1,2-a]pyrazin-8-yl)carbamate XIX was reacted with 2-Bis(tert-butoxycarbonyl)amino-6-bromopyrazine XIV according to the analogous method of CHEMISTRY A as described in Example 2 to provide the desired compound (R)-tert-butyl (6-(6-(bis(tert-butoxycarbonyl)amino)pyrazin-2-yl)imidazo[1,2-a]pyrazin-8-yl)(4-(2-(((tert-butoxycarbonyl)oxy)methyl)morpholino)phenyl)carbamate XX.

(R)-(4-(4-((6-(6-Amino-5-methylpyrazin-2-yl)imidazo[1,2-a]pyrazin-8-yl)amino)phenyl)morpholin-2-yl)methanol (3): (R)-tert-butyl (6-(6-(bis(tert-butoxycarbonyl)aminio)pyrazin-2-yl)imidazo[1,2-a]pyrazin-8-yl)(4-(2-(((tert-butoxycarbonyl)oxy)methyl)morpholino)phenyl)carbamate XX (460 mg, 0.56 mmol) in DCM was added to a round bottom flask, and TFA (1.29 ml, 16.85 mmol) was added. The reaction was partially complete after stirring ˜5 hours. Added an additional 10 eq TFA and stirred overnight, then concentrated under reduced pressure. 10% MeOH/DCM (˜100mL) and sat.aq. sodium bicarbonate were added and stirred 15 min, separated, extracted with ˜100 mL 10% MeOH/DCM. The organic layers were combined, washed with brine, dried over Na₂SO₄, filtered and concentrated under reduced pressure and dried under vacuum. The resulting solid was triturated with DCM, collected solids via filtration and dried under vacuum to give compound 3. LCMS-ESI⁺ (m/z): [M+H]⁺: 419.2. ¹H NMR (300 MHz d₆-DMSO) δ: 9.57 (s, 1H), 8.59 (s, 1H), 8.47 (s, 1H), 8.13 (d, J=1.2 Hz, 1H), 8.06-7.90 (m, 2H), 7.87 (s, 1H), 7.62 (d, J=1.1 Hz, 1H), 7.05-6.93 (m, 2H), 6.49 (s, 2H), 4.78 (t, J=5.8 Hz, 1H), 3.98-3.87 (m, 1H), 3.71-3.36 (m, 7H), 2.63 (td, J=11.7, 3.4 Hz, 1H), 2.37 (dd, J=12.1, 10.5 Hz, 1H). The corresponding (S) isomer, or racemic mixture of compounds is prepared similarly, using (S)-(4-(4-aminophenyl)morpholin-2-yl)methanol or a racemic mixture of (4-(4-aminophenyl)morpholin-2-yl)methanol, respectively, in the first step.

### Example 4

### Preparation of 6-(6-aminopyrazin-2-yl)-5-methyl-N-(4-(4-(oxetan-3-yl)piperazin-1-yl)phenyl)imidazo[1,2-a]pyrazin-8-amine (4)

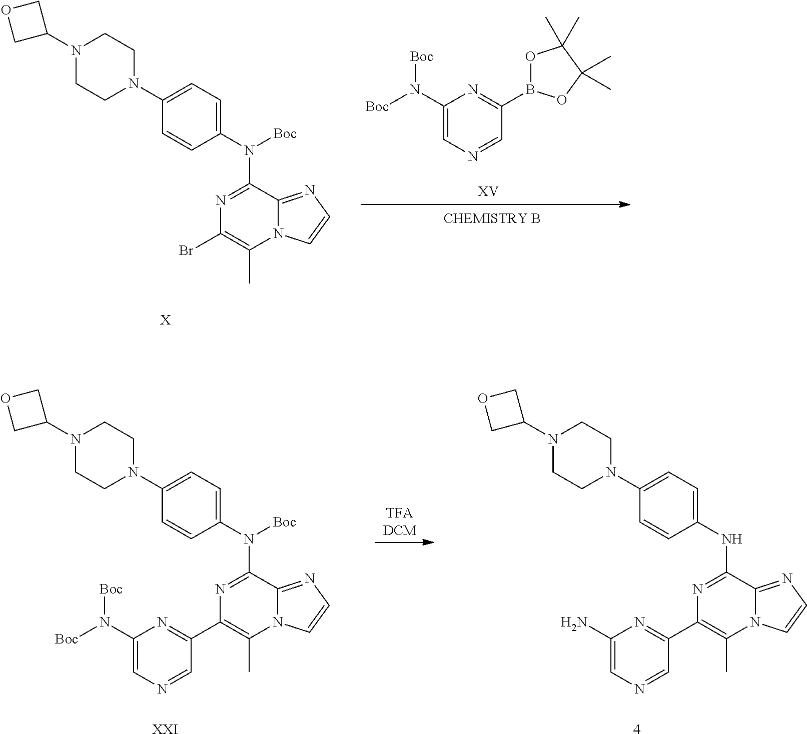

tert-Butyl (6-(6-(bis(tert-butoxycarbonyl)amino)pyrazin-2-yl)-5-methylimidazo[1,2-a]pyrazin-8-yl)(4-(4-(oxetan-3-yl)piperazin-1-yl)phenyl)carbamate XXI: tert-Butyl (6-bromo-5-methylimidazo[1,2-a]pyrazin-8-yl)(4-(4-(oxetan-3-yl)piperazin-1-yl)phenyl)carbamate X was reacted with XV according to the methods of CHEMISTRY B as described in Example 2 to provide the desired compound XXI.

6-(6-aminopyrazin-2-yl)-5-methyl-N-(4-(4-(oxetan-3-yl)piperazin-1-yl)phenyl)imidazo[1,2-a]pyrazin-8-amine (4): The compound tert-butyl (6-(6-(bis(tert-butoxycarbonyl)amino)pyrazin-2-yl)-5-methylimidazo[1,2-a]pyrazin-8-yl)(4-(4-(oxetan-3-yl)piperazin-1-yl)phenyl)carbamate XXI was de-protected by the analogous method described in Example 2 to provide the desired compound 4. LCMS-ESI⁺ (m/z): [M+H]⁺: 458.32. ¹H NMR (300 MHz, d₆-DMSO) δ: 9.28 (s, 1H), 8.28 (s, 1H), 8.04 (s, 1H), 7.89 (d, 2H), 7.83 (s, 1H), 7.7 (s,1H), 6.91 (d, 2H), 6.46 (s, 2H), 4.6-4.4 (dt, 4H), 3.43 (m, 1H), 3.1 (t, 4H), 2.49 (s,3H), 2.4 (t,4H).

### Example 5

### Preparation of 2-(5-((6-(6-aminopyrazin-2-yl)imidazo[1,2-a]pyrazin-8-yl)amino)-2-(4-(oxetan-3-yl)piperazin-1-yl)phenoxy)ethanol (5)

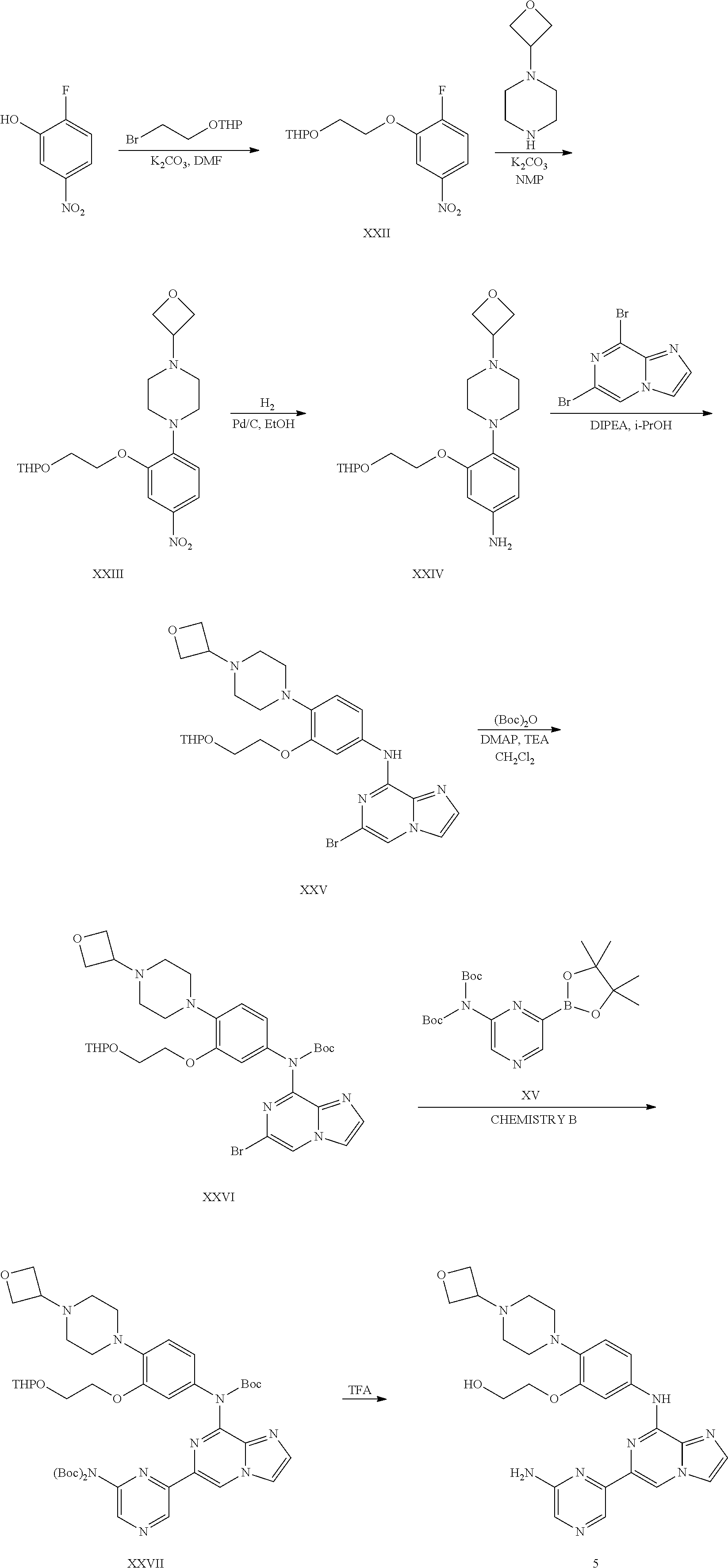

2-(2-(2-Fluoro-5-nitrophenoxy)ethoxy)tetrahydro-2H-pyran XXII: A mixture of 2-fluoro-5-nitrophenol (4 g, 25 mmol), 2-(2-bromoethoxy)tetrahydro-2H-pyran (4.4 mL, 28 mmol) and potassium carbonate (4.2 g 30 mmol) in DMF (50 mL) was stirred at 50° C. for 16 h. The reaction was cooled to room temperature, diluted with EtOAc and H₂O. The aqueous layer was separated and extracted with EtOAc. The combined organic extracts were washed with H₂O (5×'s to remove DMF) and brine and dried over sodium sulfate. The resulting residue was purified by column chromatography ISCO Rf (40 g column) eluting with a gradient of 100% hexanes—1:1 hexanes:EtOAc to provide 2-(2-(2-fluoro-5-nitrophenoxy)ethoxy)tetrahydro-2H-pyran XXII.

1-(4-Nitro-2-(2-((tetrahydro-2H-pyran-2-yl)oxy)ethoxy)phenyl)-4-(oxetan-3-yl)piperazine XXIII: A mixture of 2-(2-(2-fluoro-5-nitrophenoxy)ethoxy)tetrahydro-2H-pyran XXII (1550 mg, 5.43 mmol), 1-(oxetan-3-yl)piperazine (772 mg, 5.43 mmol) and potassium carbonate (1126.41 mg, 8.15 mmol) in NMP (6 mL) was stirred at 100° C. for 8 h. The aqueous layer was separated and extracted with EtOAc. The combined organic extracts were washed with H₂O (5× to remove NMP) and brine and dried over sodium sulfate. The resulting residue was purified by column chromatography ISCO Rf (24 g column) eluting with a gradient of 100% DCM—60:35:5 DCM:Et₂O:MeOH to provide 1-(4-nitro-2-(2-((tetrahydro-2H-pyran-2-y0oxy)ethoxy)phenyl)-4-(oxetan-3-yl)piperazine XXIII.

4-(4-(Oxetan-3-yl)piperazin-1-yl)-3-(2-((tetrahydro-2H-pyran-2-yl)oxy)ethoxy)aniline XXIV: To a suspension of 1-(4-nitro-2-(2-((tetrahydro-2H-pyran-2-yl)oxy)ethoxy)phenyl)-4-(oxetan-3-yl)piperazine XXIII (2100 mg, 5.1 mmol) in ethanol (50 mL) was added 10% Pd/C (50% wet, 390 mg dry weight) in a 500-mL Parr hydrogenation bottle. The bottle was evacuated, charged with hydrogen gas to a pressure of 50 psi and shaken at rt for 2 h on a Parr hydrogenation apparatus. The reaction mixture was filtered, and washed with ethanol. The filtrate was concentrated in vacuo to give 4-(4-(oxetan-3-yl)piperazin-1-yl)-3-(2-((tetrahydro-2H-pyran-2-yl)oxy)ethoxy)aniline XXIV.

6-Bromo-N-(4-(4-(oxetan-3-yl)piperazin-1-yl)-3-(2-((tetrahydro-2H-pyran-2 yl)oxy)ethoxy)phenyl)imidazo[1,2-a]pyrazin-8-amine XXV: To a solution of 4-(4-(oxetan-3-yl)piperazin-1-yl)-3-(2-((tetrahydro-2H-pyran-2-yl)oxy)ethoxy)aniline XXIV (619 mg, 2.17 mmol) and 6,8-dibromoimidazo[1,2-a]pyrazine (601 mg, 2.2 mmol) in IPA (15 mL) was added N,N-Diisopropylethylamine (0.95 ml, 5.43 mmol). The mixture was stirred at 110° C. for 16 h. After this time, DCM (10 mL) and sat aqueous NaHCO₃ (15 mL) were added. The aqueous layer was separated and extracted with DCM (2×10 mL). The combined organic extracts were washed with brine (10 mL) and dried over sodium sulfate. The resulting residue was purified by column chromatography ISCO Rf (24 g column) eluting with a gradient of 100% DCM—60:35:5 DCM:Et₂O:MeOH to provide 6-bromo-N-(4-(4-(oxetan-3-yl)piperazin-1-yl)-3-(2-((tetrahydro-2H-pyran-2-yl)oxy)ethoxy)phenyl)imidazo[1,2-a]pyrazin-8-amine XXV.

t-Butyl (6-bromoimidazo[1,2-a]pyrazin-8-yl(4-(4-(oxetan-3-yl)piperazin-1-yl)-3-(2-((tetrahydro-2H-pyran-2-yl)oxy)ethoxy)phenyl)carbamate XXVI: 6-Bromo-N-(4-(4-(oxetan-3-yl)piperazin-1-yl)-3-(2-((tetrahydro-2H-pyran-2-yl)oxy)ethoxy)phenyl)imidazo[1,2-a]pyrazin-8-amine XXV (1.2 g, 2.4 mmol) was reacted according to the analogous method described in Intermediate Example 1.01 (conversion of III to IV) to provide tert-butyl (6-bromoimidazo[1,2-a]pyrazin-8-yl)(4-(4-(oxetan-3-yl)piperazin-1-yl)-3-(2-((tetrahydro-2H-pyran-2-yl)oxy)ethoxy)phenyl)carbamate XXVI.

tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)-3-(2-((tetrahydro-2H-pyran-2-yl)oxy)ethoxy)phenyl)carbamate XXVII: tert-Butyl (6-bromoimidazo[1,2-a]pyrazin-8-yl)(4-(4-(oxetan-3 -yl)piperazin-1-yl)-3-(2-((tetrahydro-2H-pyran-2-yl)oxy)ethoxy)phenyl)carbamate XXVI was reacted with XV according to the methods of CHEMISTRY B as described in Example 2 to provide the desired compound tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)-3-(2-((tetrahydro-2H-pyran-2-yl)oxy)ethoxy)phenyl)carbamate XXVII.

2-(5-((6-(6-aminopyrazin-2-yl)imidazo[1,2-a]pyrazin-8-yl)amino)-2-(4-(oxetan-3-yl)piperazin-1-yl)phenoxy)ethanol (5): The compound tert-butyl (6-(6-(bis(tert-butoxycarbonyl)amino)pyrazin-2-yl)imidazo[1,2-a]pyrazin-8-yl)(4-(4-(oxetan-3-yl)piperazin-1-yl)-3-(2-((tetrahydro-2H-pyran-2-yl)oxy)ethoxy)phenyl)carbamate XXVII (313 mg, 0.35 mmol) was de-protected by the analogous method described in Example 2 to provide 2-(5-((6-(6-aminopyrazin-2-yl)imidazo[1,2-a]pyrazin-8-yl)amino)-2-(4-(oxetan-3-yl)piperazin-1-yl)phenoxy)ethanol (5). LCMS-ESI⁺ (m/z): [M+H]⁺: 504.3. ¹H NMR (300 MHz, d₆-DMSO) δ: 9.52 (s, 1H), 8.61 (s, 1H), 8.51 (s, 1H), 8.14 (d, J=1.1 Hz, 1H), 7.89 (s, 1H), 7.81 (d, J=2.3 Hz, 1H), 7.74-7.60 (m, 2H), 6.90 (d, J=8.6 Hz, 1H), 6.47 (s, 2H), 5.74 (s, 1H), 4.86-4.76 (m, 1H), 4.50 (dt, J=25.6, 6.3 Hz, 4H), 4.04 (t, J=5.1 Hz, 2H), 3.73 (q, J=5.1 Hz, 2H), 3.51-3.42 (m, 1H), 3.02 (s, 4H), 2.40 (s, 4H).

### Example 6

### Preparation of 2-((4-(4-((6-(6-aminopyrazin-2-yl)imidazo[1,2-a]pyrazin-8-yl)amino)phenyl)piperazin-1-yl)methyl)propane-1,3-diol (6)

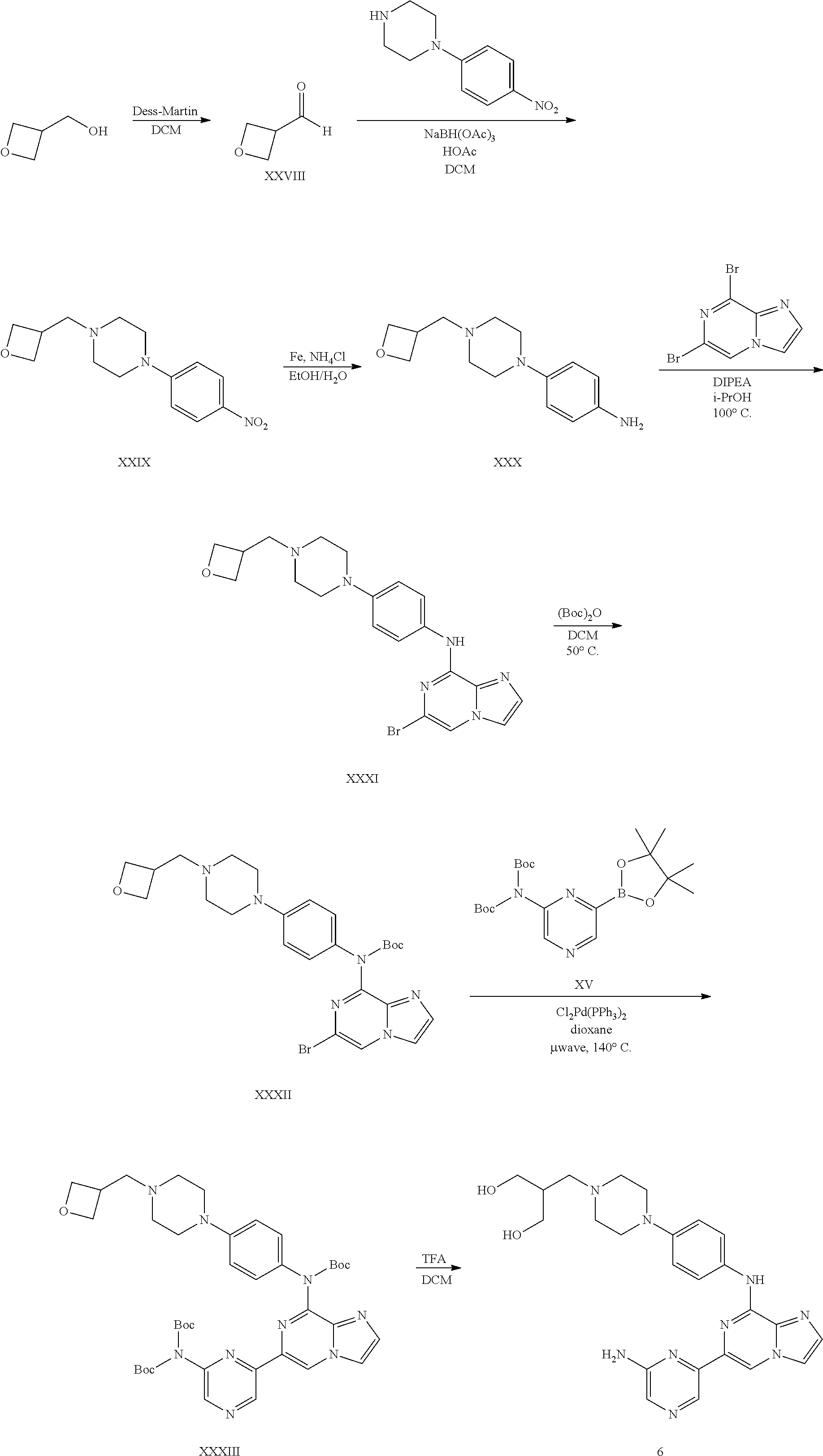

Oxetane-3-carbaldehyde XXVIII: To a round-bottomed flask equipped with a stirring bar, oxetan-3-ylmethanol (2.00 g, 22.7 mmol) was dissolved in DCM (50 mL) and Dess-Martin periodinane (10.67 g, 28.38 mmol) was added in one portion. The reaction mixture was stirred at RT overnight. The solids were filtered through celite, and washed with DCM (3 mL×5). The filtrate was removed and concentrated in vacuo and the resulting crude oxetane-3-carbaldehyde XXVIII was used in the next step directly.

1-(4-Nitrophenyl)-4-(oxetan-3-ylmethyl)piperazine XXIX: To a round-bottomed flask equipped with a stirring bar, oxetane-3-carbaldehyde XXVIII (0.977 g, 11.35 mmol), 1-(4-nitrophenyl)piperazine (1.18 g, 5.68 mmol) in DCM (100 mL), and HOAc (1.70 g, 28.38 mmol) in DCM (2 mL) were added. After 5 minutes, NaBH(OAc)₃ (24.06 g, 113.05 mmol) was added. The resulting mixture was stirred at room temperature for 2 h. Most volatiles were removed in vacuo. DCM (200 mL) was added, followed by saturated NaHCO₃ aqueous solution (20 mL), and the resulting mixture was stirred for 20 minutes. The organic phase was separated and washed with saturated NaHCO₃ aqueous solution (20 mL×3), brine (20 mL×1), dried over Na₂SO₄, filtered and solvents were removed in vacuo. The residue was passed through a silica gel column (MeOH: DCM=0:100 to 5:95 to 25:75) to provide the desired compound XXIX.

4-(4-(Oxetan-3-ylmethyl)piperazin-1-yl)aniline XXX: To a round-bottomed flask equipped with a stirring bar, were added 1-(4-nitrophenyl)-4-(oxetan-3-ylmethyl)piperazine XXIX (3.20 g, 11.54 mmol), ethanol (60 mL) and water (60 mL). Following the addition of iron (4.51 g, 80.77 mmol) and ammonium chloride (4.32 g, 80.77 mmol), the reaction mixture was heated at 80° C. for 1 h, then filtered through Celite and washed with DCM (5 mL×5). The resulting filtrate was extracted with DCM (20 mL×3). The combined organic extracts were washed with water (20 mL×2), brine (20 mL×1), dried over Na₂SO₄, and concentrated in vacuo. The desired 4-(4-(oxetan-3-ylmethyl)piperazin-1-yl)aniline XXX was obtained.

6-B romo-N-(4-(4-(oxetan-3-ylmethyl)piperazin-1-yl)phenyl)imidazo[1,2-a]pyrazin-8-amine XXXI: To a seal tube equipped with a stirring bar, 4-(4-(oxetan-3-ylmethyl)piperazin-1-yl)aniline XXX (1.19 g, 4.81 mmol), 6,8-dibromoimidazo[1,2-a]pyrazine (1.33 g, 4.81 mmol), isopropanol (24.1 mL), and diisopropylethylamine (1.37 g, 10.58 mmol) were added, and the reaction mixture was heated at 100° C. overnight. Most solvents were removed in vacuo and DCM (200 mL) was added to the mixture. The solution was washed with H₂O (20 mL×2), brine (20 mL×1), dried over Na₂SO₄, filtered and solvents were removed in vacuo. The resulting residue was passed through a silica gel column (MeOH: DCM=5:95) and light red solids were obtained as the desired compound XXXI, 0.692 g.

tert-Butyl (6-bromoimidazo[1,2-a]pyrazin-8-yl)(4-(1-(oxetan-3-ylmethyl)piperidin-4-yl)phenyl)carbamate XXXII: To a round-bottomed flask equipped with a stirring bar, were added 6-bromo-N-(4-(4-(oxetan-3-ylmethyl)piperazin-1-yl)phenyl)imidazo[1,2-a]pyrazin-8-amine XXXI (560 mg, 1.27 mmol), DCM (11 mL), di-tert-butyl dicarbonate (414.4 mg, 1.90 mmol), and triethylamine (640.5 mg, 6.33 mmol). The reaction mixture was heated at 50° C. overnight. DCM (200 mL) was added, and the resulting solution was washed with water (20 mL×2), brine (20 mL×1), dried over Na₂SO₄, filtered and solvents were removed in vacuo. Column chromatography gave the desired compound XXXII as yellow solids.

tert-Butyl (6-(6-(bis(tert-butoxycarbonyl)amino)pyrazin-2-yl)imidazo[1,2-a]pyrazin-8-yl)(4-(4-(oxetan-3-ylmethyl)piperazin-1-yl)phenyl)carbamate XXXIII: To a round-bottomed flask equipped with a stirring bar, tert-butyl (6-bromoimidazo[1,2-a]pyrazin-8-yl)(4-(4-(oxetan-3-ylmethyl)piperazin-1-yl)phenyl)carbamate XXXII (150 mg, 0.276 mmol), N, N-BisBoc 6-(4,4,5,5-tetramethyl-1,3,2-dioxaborolan-2-yl)pyrazin-2-amine XV (255.8 mg, 0.607 mmol) in DME (2.3 mL), Pd(PPh₃)₄ (16.0 mg, 0.14 mmol), Na₂CO₃ aqueous solution (1.0 N, 0.91 mL, 0.91 mmol), and DME (2 mL) were added. The mixture was heated at 75° C. for 2, then DCM (200 mL) was added and the resulting mixture was washed with water (30 mL×3), brine (30 mL×1), dried over MgSO₄, filtered, and solvents were removed in vacuo. Purification by silica gel column (MeOH: DCM=5:95) gave the desired compound XXXIII.

2-((4-(4-((6-(6-aminopyrazin-2-yl)imidazo[1,2-a]pyrazin-8-yl)amino)phenyl)piperazin-1-yl)methyl)propane-1,3-diol (6): To a solution of tert-butyl (6-(6-(bis(tert-butoxycarbonyl)amino)pyrazin-2-yl)imidazo[1,2-a]pyrazin-8-yl)(4-(4-(oxetan-3-ylmethyl)piperazin-1-yl)phenyl)carbamate XXXIII (250 mg, 0.33 mmol) in DCM (30 mL) was added TFA (940.3 mg, 8.25 mmol). The resulting mixture was stirred at room temperature for overnight. More TFA (752.2 mg, 6.60 mmol) was added and stirred at room temperature overnight. Most solvents were removed in vacuo, DCM (200 mL) and saturated NaHCO₃ aqueous solution (30 mL) were added and the resulting mixture was stirred for 30 minutes. The organic phase was separated, washed with saturated NaHCO₃ aqueous solution (20 mL×4), brine (20 mL×1). The aqueous phase was extracted with DCM (30 mL×2). The combined organic phases were washed with brine (20 mL×1), dried over Na₂SO₄, filtered, and solvents were removed in vacuo. The crude material was purified on ISCO column, MeOH: DCM=0:100 to 5:95 to 7.5:92.5 to 25:75 to elute the desired compounds. Two compounds were obtained, the first is the oxetane compound; and the other the desired compound 6. LCMS-ESI⁺ (m/z): [M+H]⁺: 476. ¹H NMR (300 MHz, d₆-DMSO) δ: 9.51 (s, 1 H), 8.60 (s, 1H), 8.49 (s, 1H), 8.14 (d, J=1.5 Hz, 1H), 7.95 (d, J=9 Hz, 2H), 7.90 (s, 1 H), 7.64 (s, 1H), 6.99 (d, J=9 Hz, 2H), 6.48 (s, 2H), 4.51 (broad S, 2H), 3.43 (d, J=6 Hz, 4H), 3.12 (broad m, 4H), 2.54 (broad m, 4H), 2.34 (d, J=7.2 Hz, 2H), 1.83 (m, 1H).

### Example 7

### Preparation of 2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol (7)

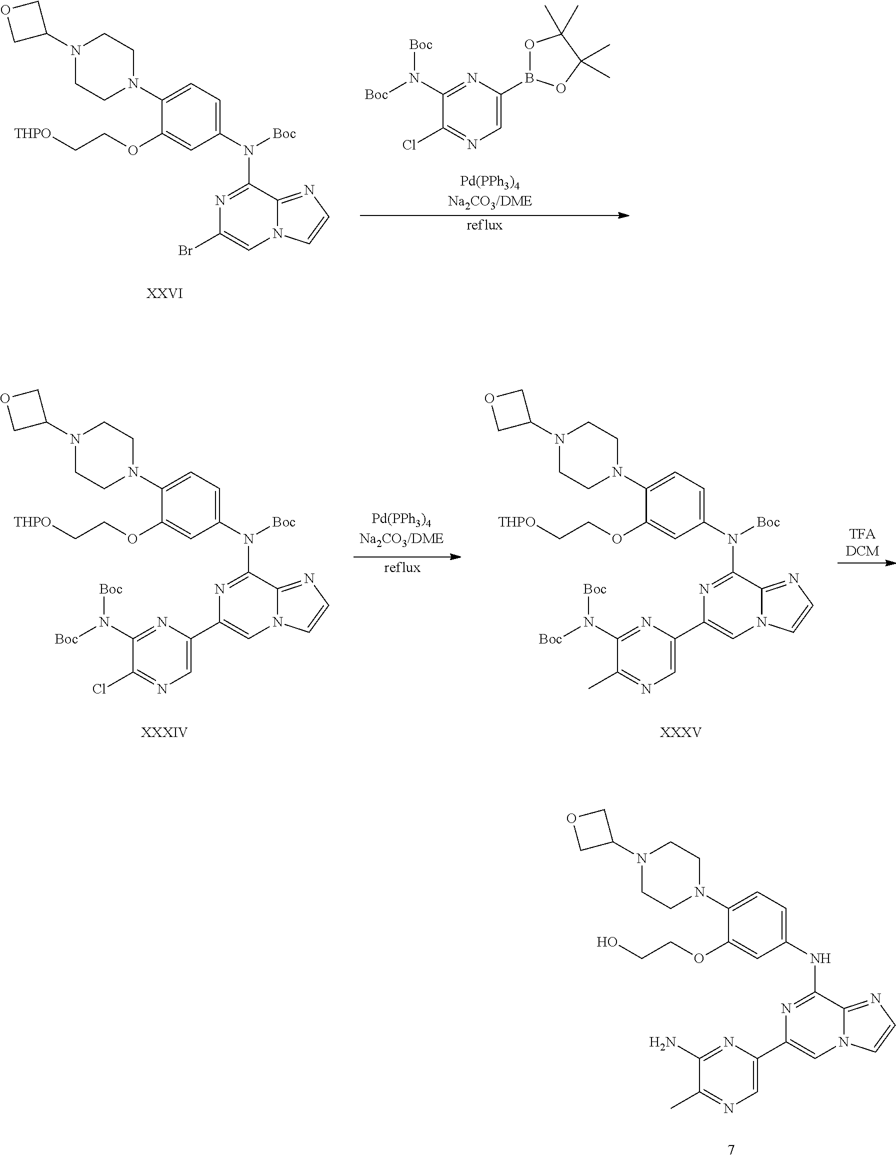

tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-chloropyrazin-2-yl)carbamate XXXIV: A flask equipped with a reflux condenser was charged with tert-butyl (6-bromoimidazo[1,2-a]pyrazin-8-yl)(4-(4-(oxetan-3-yl)piperazin-1-yl)-3-(2-((tetrahydro-2H-pyran-2-yl)oxy)ethoxy)phenyl)carbamate XXVI (prepared as described in Example 5) (352 mg, 0.52 mmol), 2-(bis-boc-amino)-3-chloro-6-(4,4,5,5-tetramethyl-1,3,2-dioxaborolan-2-yl)pyrazine (prepared by analogous method as used in Example 2 for the preparation of compound XV) (500 mg, 1.1 mmol), Pd(PPh₃)₄ (30 mg, 0.03 mmol) in sodium carbonate (1.6 mL, 1M in H₂O) and DME (4.8 mL). The mixture was heated to reflux for 1 h. The reaction was cooled to room temperature, diluted with DCM and H₂O. The aqueous layer was separated and extracted with DCM. The combined organic extracts were washed with brine, dried over sodium sulfate, filtered and concentrated under reduced pressure. The resulting residue was purified by column chromatography ISCO Rf (4 g column) eluting with a gradient of 100% DCM—100% 60/35/5 DCM/Et₂O/MeOH, appropriate fractions were combined and concentrated to provide the desired compound tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-chloropyrazin-2-yl)carbamate XXXIV.

tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-methylpyrazin-2-yl)carbamate XXXV: A microwave vial was charged with tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-chloropyrazin-2-yl)carbamate XXXIV (258 mg, 0.28 mmol), methylboronic acid (503 mg, 8.4 mmol), Pd(PPh₃)₄ (32 mg, 0.03 mmol) in sodium carbonate (0.8 mL, 1M in H₂O) and DME (2.5 mL). The mixture was heated at 150° C. for 20 min. The reaction was cooled to room temperature, diluted with DCM and H₂O. The aqueous layer was separated and extracted with DCM. The combined organic extracts were washed with brine, dried over sodium sulfate, filtered and concentrated under reduced pressure. The resulting residue was purified by column chromatography ISCO Rf (4 g column) eluting with a gradient of 100% DCM—100% 75/18/7 DCM/Et₂O/MeOH to provide the desired compound tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-methylpyrazin-2-yl)carbamate XXXV.

2-(5-((6-(6-Amino-5-methylpyrazin-2-yl)imidazo[1,2-a]pyrazin-8-yl)amino)-2-(4-(oxetan-3-yl)piperazin-1-yl)phenoxy)ethanol (7): To a solution of tert-butyl tert-butoxycarbonyl(6-(8-((tert-butoxycarbonyl)(4-(4-(oxetan-3-yl)piperazin-1-yl)-3-(2-((tetrahydro-2H-pyran-2-yl)oxy)ethoxy)phenyl)amino)imidazo[1,2-a]pyrazin-6-yl)-3-methylpyrazin-2-yl)carbamate XXXV (165 mg, 0.18 mmol) in DCM (2.2 mL) was added TFA (1.1 mL, 0.11 mmol). The mixture was stirred at rt for 16 h. The reaction was diluted with 9:1 DCM:MeOH and H₂O. The aqueous layer was separated and extracted with 9:1 DCM:MeOH. The combined organic extracts were washed with brine, dried over sodium sulfate, filtered and concentrated under reduced pressure. The resulting residue was purified by column chromatography eluting with a gradient of 100% 75/18/7 DCM/Et₂O/MeOH -100% 70/20/10 DCM/Et₂O/MeOH to provide the desired compound 2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol (7, 56 mg, 59%). LCMS-ESI⁺ (m/z): [M+H]⁺: 518.2. ¹H NMR (300 MHz, d₆-DMSO) δ: 9.49 (s, 1H), 8.56 (s, 1H), 8.44 (s, 1H), 8.13 (d, J=1.1 Hz, 1H), 7.85-7.66 (m, 2H), 7.62 (d, J=1.1 Hz, 1H), 6.90 (d, J=8.6 Hz, 1H), 6.25 (s, 2H), 4.87-4.77 (m, 1H), 4.50 (dt, J=25.2, 6.3 Hz, 4H), 4.04 (t, J=5.1 Hz, 2H), 3.74 (q, J=5.2 Hz, 2H), 3.51-3.39 (m, 1H), 3.10 -2.95 (m, 4H), 2.45 -2.35 (m, 4H), 2.34 (s, 3H). Alternatively, compound XXXIV could be taken directly to this step and similarly de-protected to provide the 5-chloropyrazine substituted analog.

### Monomesvlate and Succinate Forms

X-ray powder diffraction (XRPD) analysis of the monomesylate (MSA) and succinate forms of the compound of Example 2 herein were conducted on a diffractometer (PANanalytical XPERT-PRO, PANalytical B. V., Almelo, Netherlands) using copper radiation (Cu Ka, λ=1.5418 Å). Samples were prepared for analysis by depositing the powdered sample in the center of an aluminum holder equipped with a zero background plate. The generator was operated at a voltage of 45 kV and amperage of 40 mA. Slits used were Soller 0.02 rad., antiscatter 1.0°, and divergence. The sample rotation speed was 2 sec. Scans were performed from 2 to 40° 2-theta. Data analysis was performed by X'Pert Highscore version 2.2 c (PANalytical B. V., Almelo, Netherlands) and X'Pert data viewer version 1.2 d (PANalytical B. V., Almelo, Netherlands). The XRPD patterns for Mono MSA Forms I & II were obtained using the instrument setting as follows: 45 KV, 40 mA, Cu Kα, λ=1.5418 Å, scan range 2. −40° , step size 0.0167°, counting time: 15.875 s. The XRPD patterns for Succinate Forms I & II were obtained using the instrument setting as follows: 45 KV, 40 mA, Cu Kα, λ=1.5418 Å, scan range 2. −40°, step size 0.0084°, counting time: 95.250 s. ¹H NMR spectra of the monomesylate (MSA) and succinate forms of the compound of Example 2 were collected on a Varian 400-MR 400MHz instrument with 7620AS sample changer. The default proton parameters are as follows: spectral width: 14 to −2 ppm (6397.4 Hz); relaxation delay: 1 sec; acquisition time: 2.5559 sec; number of scans or repetitions: 8; temperature: 25 C. Samples were prepared in dimethyl sulfoxide-d6, unless otherwise stated. Off-line analysis was carried out using MNova software.

### Example 8

### 6-(6-aminopyrazin-2-yl)-N-(4-(4-(oxetan-3-yl)piperazin-1-yl)phenyl)imidazo[1,2-a]pyrazin-8-amine monomesylate Form I

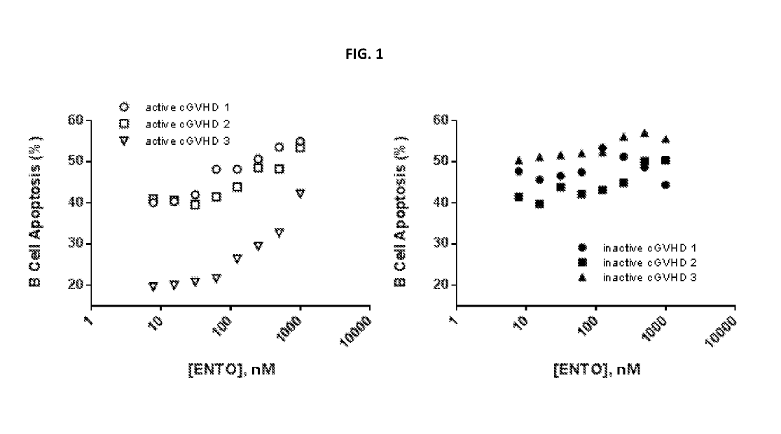

Methanesulfonic acid (MSA) salt Form I was prepared by dissolving 6-(6-aminopyrazin-2-yl)-N-(4-(4-(oxetan-3-yl)piperazin-1-yl)phenyl)imidazo[1,2-a]pyrazin-8-amine (Example 2) in 11 volumes of acetone/H₂O (36:64 vol. %) with 1 molar equivalent of methane sulfonic acid (MSA) at room temperature. The solution was then charged with 19 volumes of acetone over 1 hour and the reactor contents were stirred at room temperature overnight. XRPD analysis of 6-(6-aminopyrazin-2-yl)-N-(4-(4-(oxetan-3-yl)piperazin-1-yl)phenyl)imidazo[1,2-a]pyrazin-8-amine monomesylate Form I was conducted as described above and provided the diffraction pattern seen in FIG. 1 of US 2015/0175616 A1 (Blomgren et al.), with the peaks in the table below.

| | Pos. | Rel. Int. |
|---|---|---|
| No. | [°2Th.] | [%] |
| 1 | 19.6606 | 100 |
| 2 | 17.2746 | 93.07 |
| 3 | 17.8971 | 69.96 |
| 4 | 21.6306 | 65.74 |
| 5 | 25.7805 | 59.16 |
| 6 | 18.7593 | 51.5 |
| 7 | 13.7252 | 48.77 |
| 8 | 15.7206 | 41.91 |
| 9 | 24.7364 | 38.09 |
| 10 | 18.4345 | 36.84 |

In one embodiment 6-(6-aminopyrazin-2-yl)-N-(4-(4-(oxetan-3-yl)piperazin-1-yl)phenyl) imidazo[1,2-a]pyrazin-8-amine monomesylate Form I may be characterized by XRPD peaks 19.7 (19.6606), 17.3 (17.2746), 17.9 (17.8971), 21.6 (21.6306), and 25.8 (25.7805). In a further embodiment 6-(6-aminopyrazin-2-yl)-N-(4-(4-(oxetan-3-yl)piperazin-1-yl)phenyl)imidazo[1,2-a]pyrazin-8-amine monomesylate Form I may be characterized by XRPD peaks 19.7 (19.6606), 17.3 (17.2746), 17.9 (17.8971), and 21.6 (21.6306). In another embodiment 6-(6-aminopyrazin-2-yl)-N-(4-(4-(oxetan-3-yl)piperazin-1-yl)phenyl)imidazo[1,2-a]pyrazin-8-amine monomesylate Form I may be characterized by XRPD peaks 6.0, 6.2, 8.6, and 9.6.

NMR Analysis of 6-(6-aminopyrazin-2-yl)-N-(4-(4-(oxetan-3-yl)piperazin-1-yl)phenyl)imidazo[1,2-a]pyrazin-8-amine Mono MSA Salt Form I, conducted as described above, provided:

¹H NMR (400 MHz, DMSO-d₆) δ 10.57 (s, 1H), 9.60 (s, 1H), 8.62 (s, 1H), 8.47 (s, 1H), 8.17 (d, J=1.2 Hz, 1H), 8.03-7.96 (m, 2H), 7.90 (s, 1H), 7.69 (d, J=1.2 Hz, 1H), 7.09 (d, J=9.0 Hz, 2H), 4.78 (p, J=8.0 Hz, 4H), 4.49 (m, 1H), 4.00-2.8 (m, 10H), 2.32 (s, 3H).

### Example 9

### 6-(6-aminopyrazin-2-yl)-N-(4-(4-(oxetan-3-yl)piperazin-1-yl)phenyl)imidazo[1,2-a]pyrazin-8-amine monomesylate Form II

6-(6-aminopyrazin-2-yl)-N-(4-(4-(oxetan-3-yl)piperazin-1-yl)phenyl)imidazo[1,2-a]pyrazin-8-amine Mono MSA Salt Form II was prepared by drying 6-(6-aminopyrazin-2-yl)-N-(4-(4-(oxetan-3-yl)piperazin-1-yl)phenyl)imidazo[1,2-a]pyrazin-8-amine Mono MSA Salt Form I (Example 8) in a vacuum oven at ˜40° C. with a N₂ purge.

XRPD analysis of 6-(6-aminopyrazin-2-yl)-N-(4-(4-(oxetan-3-yl)piperazin-1-yl)phenyl)imidazo[1,2-a]pyrazin-8-amine monomesylate Form II was conducted as described above and provided the diffraction pattern seen in FIG. 5 of US 2015/0175616 A1 (Blomgren et al.), with the peaks in the table below.

| | Pos. | Rel. Int. |
|---|---|---|
| No. | [°2Th.] | [%] |
| 1 | 17.2698 | 100 |
| 2 | 25.1384 | 67.84 |
| 3 | 20.4423 | 63.66 |
| 4 | 19.5732 | 62.11 |
| 5 | 18.5264 | 50.36 |
| 6 | 17.7884 | 50.07 |
| 7 | 21.6273 | 45.52 |
| 8 | 15.2397 | 44 |
| 9 | 6.855 | 35.01 |
| 10 | 13.65 | 26 |

In one embodiment 6-(6-aminopyrazin-2-yl)-N-(4-(4-(oxetan-3-yl)piperazin-1-yl)phenyl)imidazo[1,2-a]pyrazin-8-amine monomesylate Form II may be characterized by XRPD peaks 17.3 (17.2698), 25.1 (25.1384), 20.4 (20.4423), 19.6 (19.5732), and 18.5 (18.5264). In an additional embodiment 6-(6-aminopyrazin-2-yl)-N-(4-(4-(oxetan-3-yl)piperazin-1-yl)phenyl)imidazo[1,2-a]pyrazin-8-amine monomesylate Form II may be characterized by XRPD peaks 17.3 (17.2698), 25.1 (25.1384), 20.4 (20.4423), and 19.6 (19.5732). In another embodiment 6-(6-aminopyrazin-2-yl)-N-(4-(4-(oxetan-3-yl)piperazin-1-yl)phenyl)imidazo[1,2-a]pyrazin-8-amine monomesylate Form II may be characterized by XRPD peaks 6.1, 6.9, 11.0, and 13.6.

NMR Analysis of 6-(6-aminopyrazin-2-yl)-N-(4-(4-(oxetan-3-yl)piperazin-1-yl)phenyl)imidazo[1,2-a]pyrazin-8-amine Mono MSA Salt Form II, conducted as described above, provided:

¹H NMR (400 MHz, DMSO-d₆) δ 9.61 (s, 1H), 8.63 (s, 1H), 8.47 (s, 1H), 8.19 (d, J=1.2 Hz, 1H), 8.02-7.95 (m, 2H), 7.91 (s, 1H), 7.72 (d, J=1.2 Hz, 1H), 7.13-7.06 (m, 2H), 4.85-4.72 (m, 4H), 4.53-4.45 (m, 1H), 4.30-2.75 (m, 10H), 2.34 (s, 3H).

### Example 10

### 6-(6-aminopyrazin-2-yl)-N-(4-(4-(oxetan-3-yl)piperazin-1-yl)phenyl)imidazo[1,2-a]pyrazin-8-amine succinate Form I

6-(6-aminopyrazin-2-yl)-N-(4-(4-(oxetan-3-yl)piperazin-1-yl)phenyl)imidazo[1,2-a]pyrazin-8-amine Succinate Form I was prepared by first dissolving 1.6 mol. eq. of succinic acid in THF, and then charging the acidic solution to 6-(6-aminopyrazin-2-yl)-N-(4-(4-(oxetan-3-yl)piperazin-1-yl)phenyl)imidazo[1,2-a]pyrazin-8-amine. The material was then stirred at room temperature with a magnetic stir bar overnight.

XRPD analysis of 6-(6-aminopyrazin-2-yl)-N-(4-(4-(oxetan-3-yl)piperazin-1-yl)phenyl)imidazo[1,2-a]pyrazin-8-amine succinate Form I was conducted as described above and provided the peaks in the table below.

| | Pos. | Rel. Int. |
|---|---|---|
| No. | [°2Th.] | [%] |
| 1 | 16.5 | 100 |
| 2 | 24.5 | 38.64 |
| 3 | 17.7 | 9.27 |
| 4 | 28.4 | 8.68 |
| 5 | 21.8 | 7.57 |
| 6 | 8.0 | 6.53 |
| 7 | 23.1 | 4.59 |
| 8 | 12.1 | 4.38 |
| 9 | 8.3 | 3.78 |
| 10 | 27.1 | 3.65 |

In one embodiment 6-(6-aminopyrazin-2-yl)-N-(4-(4-(oxetan-3-yl)piperazin-1-yl)phenyl)imidazo[1,2-a]pyrazin-8-amine Succinate Form I may be characterized by XRPD peaks 16.5, 24.5, 17.7, 28.4, and 21.8. In another embodiment 6-(6-aminopyrazin-2-yl)-N-(4-(4-(oxetan-3-yl)piperazin-1-yl)phenyl)imidazo[1,2-a]pyrazin-8-amine Succinate Form I may be characterized by XRPD peaks 16.5, 24.5, 8.0 and 8.3.

NMR Analysis of 6-(6-aminopyrazin-2-yl)-N-(4-(4-(oxetan-3-yl)piperazin-1-yl)phenyl)imidazo[1,2-a]pyrazin-8-amine Succinate Form I, conducted as described above, provided:

¹H NMR (400 MHz, DMSO-d₆) δ 12.12 (s, 2H), 9.48 (s, 1H), 8.59 (s, 1H), 8.48 (s, 1H), 8.12 (d, J=1.1 Hz, 1H), 7.97-7.86 (m, 3H), 7.62 (d, J=1.1 Hz, 1H), 7.01-6.94 (m, 2H), 6.45 (s, 2H), 4.55 (t, J=6.5 Hz, 2H), 4.46 (t, J=6.1 Hz, 2H), 3.49-3.38 (m, 1H), 3.13 (t, J=4.9 Hz, 4H), 2.40 (s, 10H).

The process for preparing 6-(6-aminopyrazin-2-yl)-N-(4-(4-(oxetan-3-yl)piperazin-1-yl)phenyl)imidazo[1,2-a]pyrazin-8-amine succinate Form I was also repeated using IPA, Acetone, and 2-MeTHF as solvents.

### Example 11

### 6-(6-aminopyrazin-2-yl)-N-(4-(4-(oxetan-3-yl)piperazin-1-yl)phenyl)imidazo[1,2-a]pyrazin-8-amine succinate Form II

6-(6-aminopyrazin-2-yl)-N-(4-(4-(oxetan-3-yl)piperazin-1-yl)phenyl)imidazo[1,2-a]pyrazin-8-amine free base was charged with 10.0 parts 2-propanol, followed by rapid agitation, to form a slurry. A separate solution of succinic acid (0.43 parts, 1.6 mol eq.) in 2-propanol (15 parts) was prepared at ambient temperature and was added to the slurry. The resulting slurry was then agitated at ambient temperature for about 1 day. Another solution of succinic acid (0.09 parts, 0.3 mol eq.) in 2-propanol (3 parts) was added to the slurry and the resulting slurry was agitated at ambient temperature for about two days. An additional solution of succinic acid (0.27 parts, 1.0 mol eq.) in 2-propanol (8 parts) was prepared at ambient temperature and added to the slurry and the resulting slurry was agitated for about 2 days. Then the content temperature was adjusted to 40° C. and the slurry was agitated for about two hours. The content was then returned to ambient temperature and agitated for about 16 hours. The resulting slurry was then filtered, rinsed with 2-propanol (7.0 parts), and dried at 60° C.

XRPD analysis of 6-(6-aminopyrazin-2-yl)-N-(4-(4-(oxetan-3-yl)piperazin-1-yl)phenyl)imidazo[1,2-a]pyrazin-8-amine succinate Form II was conducted as described above and provided the peaks in the table below.

| | Pos. | Rel. Int. |
|---|---|---|
| No. | [°2Th.] | [%] |
| 1 | 24.9821 | 100 |
| 2 | 16.3186 | 38.39 |
| 3 | 21.952 | 18.44 |
| 4 | 7.8958 | 17.62 |
| 5 | 7.5828 | 6.9 |
| 6 | 28.5998 | 6.52 |
| 7 | 11.3329 | 5.73 |
| 8 | 30.8568 | 5.48 |
| 9 | 28.0273 | 5.21 |
| 10 | 21.5026 | 4.73 |

In one embodiment 6-(6-aminopyrazin-2-yl)-N-(4-(4-(oxetan-3-yl)piperazin-1-yl)phenyl)imidazo[1,2-a]pyrazin-8-amine Succinate Form II may be characterized by XRPD peaks 25.0 (24.9821), 16.3 (16.3186), 22.0 (21.952), 7.9 (7.8958), and 7.6 (7.5828). In a further embodiment 6-(6-aminopyrazin-2-yl)-N-(4-(4-(oxetan-3-yl)piperazin-1-yl)phenyl)imidazo[1,2-a]pyrazin-8-amine Succinate Form II may be characterized by XRPD peaks 25.0 (24.9821), 16.3 (16.3186), 7.9 (7.8958), and 7.6 (7.5828).

NMR Analysis of 6-(6-aminopyrazin-2-yl)-N-(4-(4-(oxetan-3-yl)piperazin-1-yl)phenyl)imidazo[1,2-a]pyrazin-8-amine Succinate Form II, conducted as described above, provided:

¹H NMR (400 MHz, DMSO-d₆) δ 12.13 (s, 2H), 9.48 (s, 1H), 8.58 (s, 1H), 8.47 z (s, 1H), 8.12 (d, J=1.1 Hz, 1H), 7.97-7.86 (m, 3H), 7.62 (d, J=1.1 Hz, 1H), 7.02-6.94 (m, 2H), 6.45 (s, 2H), 4.55 (t, J=6.5 Hz, 2H), 4.46 (t, J=6.0 Hz, 2H), 3.44 (p, J=6.3 Hz, 1H), 3.17-3.10 (m, 4H), 2.40 (s, 10H), 1.02 (d, J=6.1 Hz, 2H).

### Biological Examples

### Example 12

### High Throughput Syk Biochemical Assay

Syk activity was measured using KinEASE (Cisbio), a time-resolved fluorescence resonance energy transfer (TR-FRET) immunoassay. In this assay, Syk-catalyzes the phosphorylation of a XL665-labeled peptide substrate. Europium conjugated phospho-tyrosine specific antibody binds the resulting phosphorylated peptide. Formation of phosphorylated peptide is quantified by TR-FRET with Europium as the donor and XL665 the acceptor in a 2-step endpoint assay. In brief, test compounds serially diluted in DMSO were delivered into Corning white, low volume, non-binding 384 well plates using the Echo 550 acoustic liquid dispenser (Labcyte®). Syk enzyme and substrates were dispensed into assay plates using a Multi-Flo (Bio-Tek Instruments). The standard 5 □L reaction mixture contained 20 μM ATP, 1 μM biotinylated peptide, 0.015 nM of Syk in reaction buffer (50 mM Hepes, pH 7.0, 0.02% NaN₃, 0.1% BSA, 0.1 mM Orthovanadate, 5 mM MgCl₂, 1mM DTT, 0.025% NP-40). After 30 minutes of incubation at room temperature, 5 μL of Stop and Detect Solution (1:200 Europium Cryptate labeled anti-phosphorylated peptide antibody solution and 125 nM strepavidin-XL665 Tracer in a 50 mM Hepes pH 7.0 detection buffer containing sufficient EDTA) was added. The plate was then further incubated for 120 minutes at room temperature and read using an Envision 2103 Multilabeled reader (PerkinElmer) with excitation/emission/FRET emission at 340 nm/615 nm/665 nm, respectively. Fluorescence intensities at 615nm and 665nm emission wavelengths were expressed as a ratio (665 nm/615 nm). Percent inhibition was calculated as follows: % Inhibition=100×(Ratio_{Sample}−Ratio_{0% inhibition})/(Ratio_{100% Inhibition}−Ratio_{0% Inhibition}) where 0.1% DMSO (0% inhibition) was the negative control and 1 μM K252a (100% inhibition) was used as the positive control. Activity of the compounds of Examples 1-7 are provided in the following table, demonstrating the compounds are Syk inhibitors with IC₅₀ below 50 nM.

| | Syk |
|---|---|
| | IC₅₀ |
| Example No.: Compound Name | (nM) |
| Ex.-1: 6-(6-amino-5-methylpyrazin-2-yl)-N-(4-(4-(oxetan-3- | 6.2 |
| yl)piperazin-1-yl)phenyl)imidazo[1,2-a]pyrazin-8-amine | |
| Ex.-2: 6-(6-aminopyrazin-2-yl)-N-(4-(4-(oxetan-3-yl)piperazin- | 13.5 |
| 1-yl)phenyl)imidazo[1,2-a]pyrazin-8-amine | |
| Ex.-3: (R)-(4-(4-((6-(6-aminopyrazin-2-yl)imidazo[1,2-a]pyrazin-8- | 13.3 |
| yl)amino)phenyl)morpholin-2-yl)methanol | |
| Ex.-4: 6-(6-aminopyrazin-2-yl)-5-methyl-N-(4-(4-(oxetan-3- | 44 |
| yl)piperazin-1-yl)phenyl)imidazo[1,2-a]pyrazin-8-amine | |
| Ex.-5: 2-(5-((6-(6-aminopyrazin-2-yl)imidazo[1,2-a]pyrazin- | 12.2 |
| 8-yl)amino)-2-(4-(oxetan-3-yl)piperazin-1-yl)phenoxy)ethanol | |
| Ex.-6: 2-((4-(4-((6-(6-aminopyrazin-2-yl)imidazo[1,2-a]pyrazin- | 14.5 |
| 8-yl)amino)phenyl)piperazin-1-yl)methyl)propane-1,3-diol | |
| Ex.-7: 2-(5-((6-(6-amino-5-methylpyrazin-2-yl)imidazo[1,2- | 8.7 |
| a]pyrazin-8-yl)amino)-2-(4-(oxetan-3-yl)piperazin-1- | |
| yl)phenoxy)ethanol | |

### Example 13

### 384-well HTBS Whole Blood CD63 Basophil Assay

Syk activity was assessed in relation to reduced activation of basophils as measured by the expression of CD63 in a human whole blood basophil cellular assay (25% blood). Basophil activation was measured in human whole blood using the Flow CAST kit (Buhlmann Laboratories AG, Baselstrasse, Switzerland) following the protocol provided by the manufacturer with minor modifications. Fresh human whole blood in heparin was collected and delivered same day (AllCells, Emeryville, Calif.). Whole blood samples were incubated with either DMSO (1% final) or serial diluted compounds in DMSO for 60 minutes at 37° C. Basophils were activated using the anti-FceRI mAb and stained with anti-CD63-FITC and anti-CCR3-PE for 20 minutes at 37° C. (per well: 50 μL of whole blood was mixed with 113 μL of stimulation buffer, 8.5 μL anti-FceRI mAb, 8.5 μL Ab stain CCR3-PE/CD63-FITC). Cells were centrifuged at 1000×g for 18 minutes and 150 μL/well of supernatant removed. Red blood cells were lysed and cells fixed by 2 rounds of cell lysing: resuspending cell pellets with 150 μL/well 1× lysis buffer, incubating at room temperature for 10 minutes, and collecting cell pellets by centrifuging for 1200 rpms for 5 minutes. Cells were washed with 150 μL/well wash buffer twice, and resuspended in a final volume of 75 μL/well of wash buffer for either immediate flow cytometery analysis or overnight incubation at 4° C. followed by flow cytometry analysis. Degranulation (basophil activation) was detected by CD63 surface expression on CCR3 positive cells. The percent CD63 positive cells within the gated basophil population were determined and normalized to the DMSO (negative control) and control compound (positive control). Activity of the compounds of Examples 1-7 are provided in the following table, demonstrating the compounds are effective in reducing the activation of basophils, with EC₅₀ below 200 nM.

| | CD63 |
|---|---|
| | EC₅₀ |
| Example No.: Compound Name | (nM) |
| Ex.-1: 6-(6-amino-5-methylpyrazin-2-yl)-N-(4-(4-(oxetan-3- | 51 |
| yl)piperazin-1-yl)phenyl)imidazo[1,2-a]pyrazin-8-amine | |
| Ex.-2: 6-(6-aminopyrazin-2-yl)-N-(4-(4-(oxetan-3-yl)piperazin-1- | 80 |
| yl)phenyl)imidazo[1,2-a]pyrazin-8-amine | |
| Ex.-3: (R)-(4-(4-((6-(6-aminopyrazin-2-yl)imidazo[1,2-a]pyrazin-8- | 63 |
| yl)amino)phenyl)morpholin-2-yl)methanol | |
| Ex.-4: 6-(6-aminopyrazin-2-yl)-5-methyl-N-(4-(4-(oxetan-3- | 157 |
| yl)piperazin-1-yl)phenyl)imidazo[1,2-a]pyrazin-8-amine | |
| Ex.-5: 2-(5-((6-(6-aminopyrazin-2-yl)imidazo[1,2-a]pyrazin-8- | 120 |
| yl)amino)-2-(4-(oxetan-3-yl)piperazin-1-yl)phenoxy)ethanol | |
| Ex.-6: 2-((4-(4-((6-(6-aminopyrazin-2-yl)imidazo[1,2-a]pyrazin-8- | 128 |
| yl)amino)phenyl)piperazin-1-yl)methyl)propane-1,3-diol | |
| Ex.-7: 2-(5-((6-(6-amino-5-methylpyrazin-2-yl)imidazo[1,2- | 167 |
| a]pyrazin-8-yl)amino)-2-(4-(oxetan-3-yl)piperazin-1- | |
| yl)phenoxy)ethanol | |

### Example 14

### Kinetic Solubility

The kinetic solubility of compounds in phosphate buffer at pH 7.4 was assessed. The compounds to be tested were dissolved in dimethylsulfoxide at a 10 mM concentration. Stock samples were diluted, 3 μl with 297 μl of the phosphate buffer at pH 7.4 (DulBecco's phosphate buffered saline (Sigma-Aldrich D8662), overall molarity is 0.149 M and pH 7.43). The samples were then incubated for 24 hours at 37° C. with shaking, the centrifuged and an aliquot taken and tested relative to a known standard concentration of 0.1 mM. The kinetic solubility of the compounds of Examples 1-7 are provided in the following table, demonstrating the compounds have kinetic solubility at pH 7.4 of greater than 90 p.M.

| | Solubility |
|---|---|
| | pH 7.4 |
| Example No.: Compound Name | (μM) |
| Ex.-1: 6-(6-amino-5-methylpyrazin-2-yl)-N-(4-(4-(oxetan-3- | 95 |
| yl)piperazin-1-yl)phenyl)imidazo[1,2-a]pyrazin-8-amine | |
| Ex.-2: 6-(6-aminopyrazin-2-yl)-N-(4-(4-(oxetan-3- | 95 |
| yl)piperazin-1-yl)phenyl)imidazo[1,2-a]pyrazin-8-amine | |
| Ex.-3: (R)-(4-(4-((6-(6-aminopyrazin-2-yl)imidazo[1,2- | 91 |
| a]pyrazin-8-yl)amino)phenyl)morpholin-2-yl)methanol | |
| Ex.-4: 6-(6-aminopyrazin-2-yl)-5-methyl-N-(4-(4-(oxetan-3- | 100 |
| yl)piperazin-1-yl)phenyl)imidazo[1,2-a]pyrazin-8-amine | |
| Ex.-5: 2-(5-((6-(6-aminopyrazin-2-yl)imidazo[1,2-a]pyrazin- | 97 |
| 8-yl)amino)-2-(4-(oxetan-3-yl)piperazin-1-yl)phenoxy)ethanol | |
| Ex.-6: 2-((4-(4-((6-(6-aminopyrazin-2-yl)imidazo[1,2- | 99 |
| a]pyrazin-8-yl)amino)phenyl)piperazin-1- | |
| yl)methyl)propane-1,3-diol | |

### Example 15

### Human Hepatocyte Stability Assay

The human hematocyte stability of the compounds as predicted hepatocyte clearance in L/hr/kg was assessed. Compounds to be tested were diluted to 200 μM (4 μl of 10 mM DMSO stock into 196 μl ACN:H₂O (50:50). Propranolol was used as a positive control, and buffer only without hepatocytes as 0% control. These were further diluted 4 μl with 891 μl KHB buffer (InVitroGRO catalog number Z99074) to provide 2× dosing solution. To each well of 24 well plate, 250 μl of the 2× dosing solution was added to each well with 250 μl of hepatocytes cells (1×10⁶ viable cells/ml per well) or KHB for control samples to achieve a final compound concentration of 1 μM during incubation. The final solvent concentration was 0.01% DMSO and 0.25% ACN. The culture plate was placed on a rocker and incubated at 37° C., 5% CO₂. Samples were collected at time 0, 1, 3, and 6 hours. The loss of parent compound was determined using LC-MS methods against a standard curve. Activity of the compounds of Examples 1-7 are provided in the following table, showing hepatocyte clearance of about 0.12 L/hr/kg or less.

| | Hheps |
|---|---|
| | CL |
| Example No.: Compound Name | (L/hr/kg) |
| Ex.-1: 6-(6-amino-5-methylpyrazin-2-yl)-N-(4-(4-(oxetan-3- | 0.12 |
| yl)piperazin-1-yl)phenyl)imidazo[1,2-a]pyrazin-8-amine | |
| Ex.-2: 6-(6-aminopyrazin-2-yl)-N-(4-(4-(oxetan-3- | 0.055 |
| yl)piperazin-1-yl)phenyl)imidazo[1,2-a]pyrazin- | |
| 8-amine | |
| Ex.-3: (R)-(4-(4-((6-(6-aminopyrazin-2-yl)imidazo[1,2- | 0.09 |
| a]pyrazin-8-yl)amino)phenyl)morpholin-2-yl)methanol | |
| Ex.-4: 6-(6-aminopyrazin-2-yl)-5-methyl-N-(4-(4-(oxetan-3- | 0.08 |
| yl)piperazin-1-yl)phenyl)imidazo[1,2-a]pyrazin-8-amine | |
| Ex.-5: 2-(5-((6-(6-aminopyrazin-2-yl)imidazo[1,2-a]pyrazin-8- | 0.07 |
| yl)amino)-2-(4-(oxetan-3-yl)piperazin-1-yl)phenoxy)ethanol | |
| Ex.-6: 2-((4-(4-((6-(6-aminopyrazin-2-yl)imidazo[1,2- | 0.08 |
| a]pyrazin-8-yl)amino)phenyl)piperazin-1- | |
| yl)methyl)propane-1,3-diol | |
| Ex.-7: 2-(5-((6-(6-amino-5-methylpyrazin-2-yl)imidazo[1,2- | 0.05 |
| a]pyrazin-8-yl)amino)-2-(4-(oxetan-3-yl)piperazin-1- | |
| yl)phenoxy)ethanol | |

### Example 16

### Comparison to known Syk Inhibitors

The assays of Examples 8-11 were used to compare the compounds as described herein with compounds known in the art. The data comparing the compounds of Examples 1-7 to previously described compounds is provided in the following table. From these results, it is clear that compounds as described herein are desirable as Syk inhibitors, with improved Syk and CD63 activity relative to the known compounds, improved kinetic solubility (at least about 9-fold more soluble) and hepatocyte clearance (at least about 2-fold less clearance). As such, the combination of improved Syk and CD63 inhibitory activity with improved kinetic solubility and clearance provides compounds that are expected to be effective at treating diseases as described herein with improved pharmacokinetic properties.

| | Syk | CD63 | Solubility | Hheps |
|---|---|---|---|---|
| | IC₅₀ | IC₅₀ | pH 7.4 | CL |
| Compound Name | (nM) | (nM) | (μM) | (units) |
| Ex.-1: 6-(6-amino-5-methylpyrazin- | 6.2 | 51 | 95 | 0.12 |
| 2-yl)-N-(4-(4-(oxetan-3-yl)piperazin- | | | | |
| 1-yl)phenyl)imidazo[1,2-a]pyrazin-8- | | | | |
| amine | | | | |
| Ex.-2: 6-(6-aminopyrazin-2-yl)-N- | 13.5 | 80 | 95 | 0.055 |
| (4-(4-(oxetan-3-yl)piperazin-1- | | | | |
| yl)phenyl)imidazo[1,2-a]pyrazin-8- | | | | |
| amine | | | | |
| Ex.-3: (R)-(4-(4-((6-(6- | 13.3 | 63 | 91 | 0.09 |
| aminopyrazin-2-yl)imidazo[1,2- | | | | |
| a]pyrazin-8- | | | | |
| yl)amino)phenyl)morpholin-2- | | | | |
| yl)methanol | | | | |
| Ex.-4: 6-(6-aminopyrazin-2-yl)-5- | 44 | 157 | 100 | 0.08 |
| methyl-N-(4-(4-(oxetan-3- | | | | |
| yl)piperazin-1- | | | | |
| yl)phenyl)imidazo[1,2-a]pyrazin-8- | | | | |
| amine | | | | |
| Ex.-5: 2-(5-((6-(6-aminopyrazin-2- | 12.2 | 120 | 97 | 0.07 |
| yl)imidazo[1,2-a]pyrazin-8- | | | | |
| yl)amino)-2-(4-(oxetan-3- | | | | |
| yl)piperazin-1-yl)phenoxy)ethanol | | | | |
| Ex.-6: 2-((4-(4-((6-(6-aminopyrazin- | 14.5 | 128 | 99 | 0.08 |
| 2-yl)imidazo[1,2-a]pyrazin-8- | | | | |
| yl)amino)phenyl)piperazin-1- | | | | |
| yl)methyl)propane-1,3-diol | | | | |
| Ex.-7: 2-(5-((6-(6-amino-5- | 8.7 | 167 | nd | 0.05 |
| methylpyrazin-2-yl)imidazo[1,2- | | | | |
| a]pyrazin-8-yl)amino)-2-(4-(oxetan- | | | | |
| 3-yl)piperazin-1-yl)phenoxy)ethanol | | | | |

Known compounds:

| | | | | |
|---|---|---|---|---|
| 6-(5-aminopyridin-3-yl)-N-(4- | 31 | 101 | 5 | 0.68 |
| morpholinophenyl)imidazo[1,2- | | | | |
| a]pyrazin-8-amine | | | | |
| 6-(3-aminophenyl)-N-(3,4- | 188 | 809 | 3 | 0.24 |
| dimethoxyphenyl)imidazo[1,2- | | | | |
| a]pyrazin-8-amine | | | | |
| 6-(5-amino-6-methylpyridin-3-yl)-N- | 16 | 250 | 5 | 0.80 |
| (4-morpholinophenyl)imidazo[1,2- | | | | |
| a]pyrazin-8-amine | | | | |
| 6-(6-aminopyridin-3-yl)-N-(3,4- | 53 | 734 | 10 | 0.90 |
| dimethoxyphenyl)imidazo[1,2- | | | | |
| a]pyrazin-8-amine | | | | |

### Example 17

### Apoptosis Assay

Entospletinib (Formula I) was prepared as a 10 mM stock in dimethyl sulfoxide (DMSO). Before use, entospletinib was thawed from 10 mM DMSO stocks frozen in 0.75 mL polypropylene tubes at −20° C.

Reagents

| Reagent | Supplier | Catalog No. |
|---|---|---|
| Roswell Park Memorial Institute | Sigma | R8758 |
| (RPMI)-1640 Base Medium | | |
| Fetal Bovine Serum (FBS) | Gemini | 100-106 |
| 1X Phosphate Buffered Saline | Life Technologies | 14040 |
| (PBS^{+/+}) | | |
| 1X Phosphate Buffered Saline | Life Technologies | 14190 |
| (PBS^{-/-}) | | |
| 4-(2-hydroxyethyl)-1-piperazineethane- | Sigma | H0887 |
| sulfonic acid (HEPES) | | |
| Penicillin-Streptomycin | Sigma | P0781 |
| Dimethyl Sulfoxide (DMSO) | Sigma | D2650 |
| Human TruStain FcX ™ | BioLegend | 422302 |
| 7-AAD | BD Biosciences | 559925 |
| Annexin V Apoptosis Detection | eBioscience | 88-8007-74 |
| Kit-APC | | |
| Pacific Blue-conjugated anti-human | BioLegend | 302224 |
| CD19 antibody | | |
| Fixation Buffer | BD Biosciences | 554655 |
| 96-well Cell Culture Plates | Costar | 3596 |
| Foil Seals | ATCC | 60-2400 |

Viably-frozen peripheral blood mononuclear cells (PBMCs) from 3 subjects with active chronic graft-versus host disease (cGVHD) and 3 subjects with inactive cGVHD were plated at 1×10⁶ B cells per well of a 96-well plate in 110 μl RPMI-1640 medium supplemented with 10% FBS, 10 mM HEPES, Pen/Strep, and 2-fold serial dilutions of GS-9973 ranging from concentrations of 1.0-0.0078 μM. For untreated PBMCs, DMSO alone (the diluent used to generate entospletinib stock solution) was used in the cultures at the equivalent volume as for the 1.0 μM entospletinib treatment group. The cells were then incubated for 48 hr at 37° C. and 5% CO₂, harvested, and assessed for the frequency of apoptotic B cells by flow cytometry analysis as described below.

Flow Cytometry Analysis—Cultured PBMCs were washed in FACS wash buffer (PBS containing 2% FBS) and then resuspended in FACS wash containing Fc block (Human TruStain FcXTM Fc Receptor Blocking Solution from BioLegend, San Diego, Calif.) at the recommended concentration. Following a 15 minute incubation on ice, the cells were stained with Pacific Blue™ conjugated anti-human CD19 antibody (BioLegend, Inc.) for an additional 30 min and then washed in cold PBS, followed by a second wash with Annexin V Binding Buffer (Annexin V Apoptosis Detection Kit-APC, eBioscience, Inc.) according to the manufacturer's instructions. The cells were then resuspended in Annexin V Binding Buffer containing APC-conjugated Annexin V, and incubated in the dark for 15 min at RT. Finally, the cells were washed with cold Annexin V Binding Buffer, resuspended in cold Annexin V Binding Buffer containing 7-AAD (BD Biosciences), kept on ice, and analyzed immediately on a FACSCantoTM flow cytometer. Flow cytometry data files were analyzed using FlowJo software (version X) to identify B cells and determine the frequencies of apoptotic cells based on Annexin V and 7-AAD staining.

For each set of patient samples, B cell apoptosis induced by entospletinib at each concentration was determined by the following ratio: % Annexin V⁺/7-AAD⁻ B cells (entospletinib-treated)/% Annexin V⁺/7-AAD⁻ B cells (untreated). Statistical analysis comparing the ratios of apoptotic B cells between the active and inactive cGVHD groups was then performed using a two-tailed, non-paired Student's t-test (GraphPad Prism software, version 5). Graphic display and curve fit analysis to determine the EC₅₀ for entospletinib apoptosis-inducing activity in active cGVHD B cells was performed using GraphPad Prism software (GraphPad Software, La Jolla, Calif).

### Results

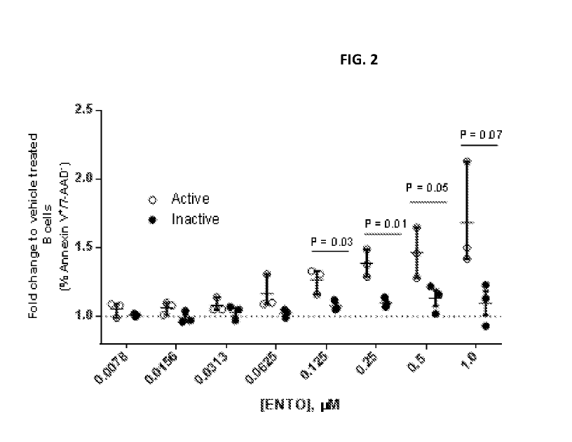

Viably frozen PBMCs from 3 subjects with active cGVHD and from 3 HSCT subjects without cGVHD were thawed and incubated with 2-fold serial dilutions of ENTO over a concentration range of 1.0-0.0078 μM for 48 hours. Apoptosis of B cells was measured and quantified by flow cytometry. The data (Table 1) are depicted in FIG. 1 for each subject sample and demonstrates that entospletinib caused apoptosis of B cells obtained from subjects with cGVHD. In one subject with cGVHD, B cells had a low level of baseline apoptosis and entospletinib caused a dose-dependent increase in B cell apoptosis. Though samples from the other two cGVHD subjects had a higher baseline level of B cell apoptosis in vitro, treatment with entospletinib also caused an increase in B cell apoptosis. The mean results from 3 donors are shown graphically in FIG. 2 as mean fold increase in B cell apoptosis relative to the vehicle control. The data demonstrate that treatment with >125 nM ENTO caused a statistically significant dose-dependent increase in B cell apoptosis in cGVHD samples versus from subjects without cGVHD.

FIG. 1 depicts the values for PBMCs from subjects with cGVHD (open circles) and without cGVHD (filled circles) treated with ETNO (7.8 nM−1.0 μM) as indicated for 48 hours. Apoptotic B cells were defined as CD19⁺ annexin V⁺7AAD⁻ cells.

FIG. 2 depicts apoptosis in human PBMCs from subjects without cGVHD (n=3, filled squares) and with cGVHD (n=3, filled circles) were treated with ENTO (7.8 nM −1 μM) as indicated for 48 hours. Apoptotic B cells were defined as CD19⁺ AnnexinV⁺ 7AAD⁻ cells and the fold induction of apoptosis over the vehicle treated samples alone is plotted. Statistics are the difference in fold-change between subjects with cGVHD and those without cGVHD.

TABLE 1
Frequency of Apoptotic B Cells in cGVHD and Inactive
cGVHD Subjects Treated with Entospletinib for 48 hours

| | Annexin V+/7AAD− | | | | | |
|---|---|---|---|---|---|---|
| | (frequency of parent) | | | | | |
| [Entospletinib], | cGVHD | cGVHD | cGVHD | Inactive | Inactive | Inactive |
| nM | #1 | #2 | #3 | #1 | #2 | #3 |
| 1000 | 55.0 | 53.5 | 42.2 | 44.4 | 50.4 | 55.6 |
| 500 | 53.6 | 48.3 | 32.7 | 48.7 | 50.2 | 57.1 |
| 250 | 50.7 | 48.6 | 29.5 | 51.3 | 44.9 | 56.2 |
| 125 | 48.2 | 43.9 | 26.4 | 53.4 | 43.2 | 52.4 |
| 62.5 | 48.2 | 41.5 | 21.6 | 47.5 | 42.2 | 52.1 |
| 31.3 | 42.0 | 39.6 | 20.8 | 46.6 | 43.9 | 51.7 |
| 15.6 | 40.4 | 40.7 | 20.0 | 45.7 | 39.8 | 51.2 |
| 7.8 | 40.1 | 41.0 | 19.6 | 47.7 | 41.5 | 50.4 |
| 0 | 36.7 | 37.8 | 19.8 | 47.8 | 41.1 | 49.4 |

Throughout this specification, various patents, patent applications and other types of publications (e.g., journal articles) are referenced. The disclosure of all patents, patent applications, and publications cited herein are hereby incorporated by reference in their entirety for all purposes.

1. A method for treating graft versus host disease in a human, the method comprising administering to the human in need thereof a pharmaceutically effective amount of a compound selected from the group consisting of the compounds of Formula (I) and Formula (H), or a pharmaceutically acceptable salt or co-crystal thereof: 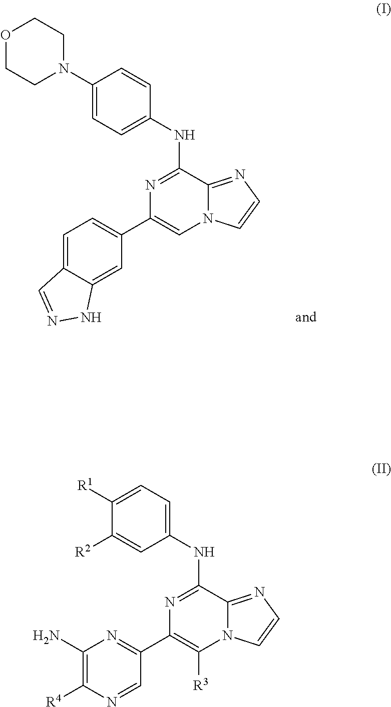
wherein, in Formula (II):
R¹ is selected from the group consisting of 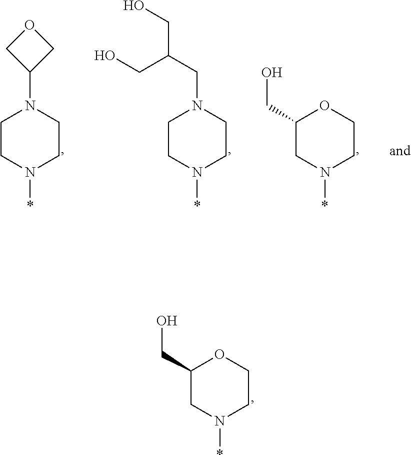
wherein * indicates the carbon atom of the indicated phenyl ring of Formula I to which R¹ is attached;
R² is H or 2-hydroxyethoxyl;
R³ is H or methyl; and
R⁴ is H or methyl.

2. A method for inhibiting the onset of symptoms of GVHD, the method comprising administering to a human recipient of a transplantation of allogenic heniatopoietic stem cells a pharmaceutically effective amount of a compound selected from the group consisting of the compounds of Formula (I) and Formula (II), or a pharmaceutically acceptable salt or co-crystal thereof: 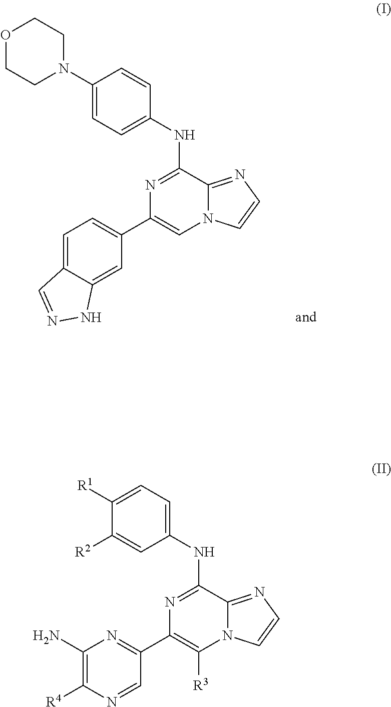
wherein, in Formula (II);
R¹ is selected from the group consisting of 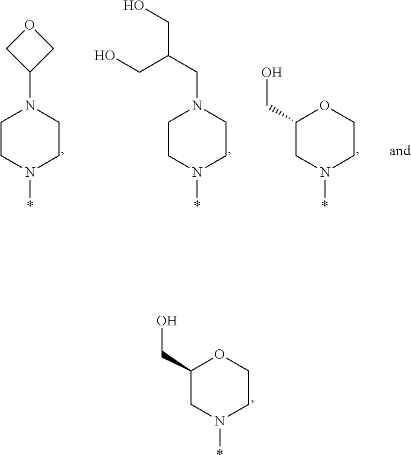
wherein * indicates the carbon atom of the indicated phenyl ring of Formula I to which R¹ is attached;
R² is H or 2-hydroxyethoxyl;
R³ is H or methyl; and
R⁴ is H or methyl.

3. The method of claim 1 wherein the compound is: 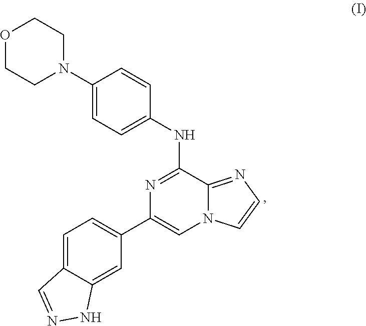 [SMILES: c1cn2cc(-c3ccc4cn[nH]c4c3)nc(Nc3ccc(N4CCOCC4)cc3)c2n1]
or a pharmaceutically acceptable salt or co-crystal thereof.

4. The method of claim 1 wherein the compound is 6-(6-amino-5-methylpyrazin-2-yl)-N-(4-(4-(oxetan-3-yl)piperazn-1-yl)imidazo[1,2-a]pyrazin-8-amine, or a pharmaceutically acceptable salt or co-crystal thereof.

5. The method of claim 1 wherein the compound is 6-(6-aminopyrazin-2-yl)-N-(4-(4-(oxetan-3-yl)piperazin-1-yl)phenyl)imidazo[1,2-a]pyrazin-8-amine, or a pharmaceutically acceptable salt or co-crystal thereof.

6. The method of claim 1 wherein the compound is (R)-(4-(4-((6-(6-aminopyrazin-2-yl)imidazo[1,2-a]pyrazin-8-yl)amino)phenyl)morpholin-2-yl)methanol, or a pharmaceutically acceptable salt or co-crystal thereof.

7. The method of claim 1 wherein the compound is 6-(6-aminopyrazin-2-yl)-5-methyl-N-(4-(4-(oxetan-3-yl)phenyl)imidazo[1,2-a]pyrazin-8-amine, or a pharmaceutically acceptable salt or co-crystal thereof.

8. The method of any of claim 1wherein the compound is 2-(5 -((6-(6-aminopyrazin-2-yl)imidazo[1,2-a]pyrazin-8-yl)amino)-2-(4-(oxetan-3-yl)piperazin-1-yl)phenoxy)ethanol, or a pharmaceutically acceptable salt or co-crystal thereof.

9. The method of claim 1 wherein the compound is 2-((4-(4-((6-(6-aminopyrazin-2-yl)imidazo[1,2-a]pyrazin-8-yl)amino)phenyl)piperazin-1-yl)methyl)propane-1,3-diol, or a pharmaceutically acceptable salt or co-crystal thereof.

10. The method of claim 1 wherein the compound is -(5-((6-(6-amino-5-methylpyrazin-2-yl)imidazo[1,2-a]pyrazin-8-yl)amino)-2-(4-(oxetan-3-yl)piperazin-1-yl)phenoxy)ethanol, or a pharmaceutically acceptable salt or co-crystal thereof.

11. The method of claim 1 wherein the GVHD is acute graft versus host disease.

12. The method of claim 1 wherein the GVHD is acute graft versus host disease.

13. The method of claim 1 further comprising administering to the human in need thereof a pharmaceutically effective amount one or more additional agents useful in the treatment of graft versus host disease.

14. The method of claim 13 wherein the one or more additional agents useful in the treatment of graft versus host disease is selected from the group of prednisone, methylprednisone, oral nonabsorbable corticosteroids, such as budesonide or beclomethasone diproprionate, immune modulators, such as cyclosporine, tacrolimus, mycophenolate mofetil, tilomisole, imuthiol, antithymocyte globulin, anti-TNF agents, azathioprine, inosine 5′-monophosphate dehydrogenase inhibitors, azodiacarbonide, bisindolyl maleimide VIII, brequinar, chlorambucil, CTLA-4Ig, corticosteroids, cyclophosphamide, deoxyspergualin, dexamethasone, glucocorticoids, leflunomide, mercaptopurine, 6-mercaptopurine, methotrexate, methylprednisolone, mizoribine, mizoribine monophosphate, muromonab CD3, mycophenolate mofetil, OKT3, rho (D) immune globin, vitamin D analogs, MC1288), daclizumab, infliximab, rituximab, tocilizumab alemtuzumab, methotrexate, antithymocyte denileukin diftitox, Campath-1H, keratinocyte growth factor, abatacept, remestemcel-L suberoylanilide hydroxamic acid, pentostatin, thalidomide, imatinib mesylate, cyclophosphamide, fludarabine, OKT3, melphalan, thiopeta, and lymphocyte immune globulin, anti-thymocyte, and globulin.

15-33. (canceled)

34. The method according to claim 1 in which the compound is a compound of Formula (II), R² is H, R³ is methyl, and R⁴ is H, or a pharmaceutically acceptable salt or co-crystal thereof.

35. The method according to claim 1 in which the compound is a compound of Formula (II), R² is H, R³ is H, and R⁴ is methyl, or a pharmaceutically acceptable salt or co-crystal thereof.

36. The method according to claim 1 in which the compound is a compound of Formula (II), R² is 2-hydroxyethoxyl, R³ is methyl, and R⁴ is H, or a pharmaceutically acceptable salt or co-crystal thereof.

37. The method according to claim 1 in which the compound is a compound of Formula (II), R² is 2-hydroxyethoxyl, R³ is methyl, and R⁴ is H, or a pharmaceutically acceptable salt or co-crystal thereof.

38. The method according to claim 1 in which the compound is a compound of Formula (II), R² is 2-hydroxyethoxyl, R³ is H, and R⁴ is methyl, or a pharmaceutically acceptable salt or co-crystal thereof.
